(19) United States
(12) Patent Application Publication
(10) Pub. No.: US 2022/0347686 A1
(43) Pub. Date: Nov. 3, 2022

(54) SIZE-BASED ASYMMETRIC NANOPORE MEMBRANE (ANM) FILTRATION FOR HIGH-EFFICIENCY EXOSOME ISOLATION, CONCENTRATION, AND FRACTIONATION

(71) Applicant: University of Notre Dame du Lac, South Bend, IN (US)

(72) Inventors: Ceming Wang, South Bend, IN (US); Hsueh-Chia Chang, South Bend, IN (US)

(21) Appl. No.: 17/760,531

(22) PCT Filed: Sep. 15, 2020
(86) PCT No.: PCT/US2020/050844

§ 371 Date: Mar. 15, 2022

Related U.S. Application Data
(60) Provisional application No. 62/901,117, filed on Sep. 16, 2019.

(51) Int. Cl. B01L 3/00

(52) U.S. Cl. CPC B01L 3/502753; B01L 2300/0896; B01L 2400/0409; B01L 2400/0418; B01L 2400/0487; B01L 2400/086

(57) ABSTRACT

Described herein is a size-based asymmetric nanopore membrane (ANM) filtration technology for high-efficiency exosome isolation, concentration, and fractionation. The ANM design prevents exosome deformation, lysing, and fusion due to the strong external force and thus significant increases the yield (up to 92%) while preserving other advantages of size-based ultrafiltration. It also offers a unique feature of being able to flush the contaminating proteins from the exosomes. It offers higher throughput, yield, sample purity, concentration factor, and more precise size fractionation than current approaches.

### CROSS-REFERENCE TO RELATED APPLICATIONS

This application claims priority to U.S. Provisional Patent Application No. 62/901,117, filed on Sep. 16, 2019, which is incorporated by reference herein in its entirety.

### FEDERALLY SPONSORED RESEARCH

This invention was made with United States government support under National Institutes of Health grant numbers 1R21CA206904-01 and HG009010-01. The United States government has certain rights in the invention.

### TECHNICAL FIELD

Described herein is a size-based asymmetric nanopore membrane (ANM) filtration technology for high-efficiency exosome isolation, concentration, and fractionation. The ANM design prevents exosome deformation, lysing, and fusion due to the strong external force and thus significant increases the yield (up to 90%) while preserving other advantages of size-based ultrafiltration. It also offers a unique feature of being able to flush the contaminating proteins from the exosomes. It offers higher throughput, yield, sample purity, concentration factor, and more precise size fractionation than current approaches.

### BACKGROUND

Liquid biopsy and other disease screening technologies are based on quantification of nucleic acid and protein biomarkers in blood. It is now realized that such molecular biomarkers are encased in nano-sized particles like microvesicles (mv), exosomes (ex), exemeres, high-density lipoproteins (HDL), low-density lipoproteins (LD), and ribonucleoprotiens (RBP). Some nanoparticles are vesicular, and some are protein-RNA complexes. Different particles originate from different cells and nitroxyl radical-containing nanoparticles (RNPs) only appear when cells are under oxidative or mechanical stress. Some have surface proteins they inherit from the cells they originate from. This heterogeneity of the molecular carriers implies that precise diagnosis of disease requires not only quantification of the nucleic acid and protein cargo but also isolation and fractionation of the nanoparticles. Similarly, nanoparticle isolation from cell cultures (cell media/supernatant) for biomarker discovery, drug testing, drug delivery and cosmetic purposes must be properly fractionated and purified so that only the nanoparticles with the proper molecular cargo are used. Nanoparticles range from 20 nm to 300 nm which makes them extremely difficult to fractionate at the high throughput required for diagnostics, biomarker discovery, drug testing, and delivery applications. Ultracentrifugation, precipitation, size exclusion chromatography, and nanofiltration can only achieve high throughput fractionation if large centrifugal force and high pressure/shear are used to force the suspension through nanofilters. The result is particle loss due to lysing. This is detrimental to diagnostics, as accurate quantification is now impossible.

Exosomes are secreted membrane enclosed vesicles (extracellular vesicles) of 50 to 200 nm diameter in all living cells [1]. Free exosomes are generated by release from endosomal derived multi-vesicular bodies (MVBs) during fusion with the plasma membrane. Most significantly, exosomes carry mRNA, miRNA, and proteins derived from their cells of origin [2-5]. The exosome-related research explosion is due, in part, to Swedish scientist Jan Lötvall from the University of Gothenburg. Exosomes had long been viewed as merely tiny trash sacs tossed from cells, but Lötvall showed in 2007 that some cells use exosomes to transfer genetic material—messenger RNAs to make proteins and microRNAs to regulate the expression of genes—between each other [3]. That discovery set scientists searching for ways that exosomes might be involved in health and disease and even be used as treatments.

The release of microvesicles has demonstrated biological relevance; these particles act as mediators of intercellular communication both within the local microenvironment as well as systemically [6]. Exosomes have been linked to a range of physiological processes, including cell proliferation [7], cancer metastasis [8], and immunomodulatory activity [9]. Given these implications, and their presence in clinical samples (plasma, urine, saliva), exosomes represent a burgeoning target for biomarker discovery with prognostic/diagnostic implications [10].

Compared with the other sources of liquid biopsies, exosomes have superiority in different aspects. First, exosomes can mirror the original cell markers by presenting specific surface proteins [11] and even their target cells [12]. These features allow easy isolation of both the tissue and target cell-specific exosomes. Second, exosomes are stable in the circulation and are found in all bodily fluids. They can hence be used as a powerful non-invasive diagnostic tool for many diseases including cancer. Third, exosomes represent their parental disease/tumor-specific RNA and protein profile, and their architecture protects circulating RNA and microRNA (miRNA) from RNase catalytic function. Therefore, exosomal nucleic acids can be utilized to find genetic signatures in patients with cancer.

The first blood-based cancer diagnostic to exploit free-floating exosomes became commercially available on January 21, 2016, which was developed by Exosome Diagnostics, Inc of Cambridge, Mass. (www.exosomedx.com/). Exosome's ExoDx Lung (ALK) test detects both exosomal RNA and ctDNA in a single-step analysis. It can boost sensitivity in detecting rare cancer mutations that are not easily detected in other liquid biopsies that rely on circulating tumor cells or ctDNA only. In a blinded study with m0/m1a NSCLC patients, whose mutation status is considered particularly difficult to assess by liquid biopsy, analyzing the exosomal RNA and ctDNA fractions improved sensitivity almost threefold (74% vs. 26%) over what could be obtained by evaluating ctDNA only [13]. Another key thread in the exosome diagnostics story is in pancreatic cancer. In a recent 250-patient study, Kalluri and colleagues found glypican-1 (GPC1), a cell membrane surface proteoglycan, specifically enriched in circulating exosomes from individuals with pancreatic cancer. GPC1 can differentiate, with 100% accuracy and sensitivity, early- and late-pancreatic cancer from benign disease [14]. That level of precision outperforms any other technologies in molecular diagnostics. The broad liquid-biopsy category also encompasses the analysis of circulating tumor cells and cell-free, circulating tumor DNA (ctDNA) derived from dying cancer cells, in addition to exosomes. Despite the increasing clinical importance of exosomes as potential biomarkers, current commercial methods of EV isolation suffer from low yield and purity as well as complicated procedures with long processing times, a simple and cheap method for isolating circulating exosomes is needed in the clinic [15].

Exosomes are also positioned to become a widespread tool for therapeutics and drug delivery. Although liposomes and nanoparticles may offer advantages for siRNA delivery over viral-based delivery systems, they exhibit low efficiency and rapid clearance from the circulation. Unlike liposomes and other synthetic drug nanoparticle carriers, exosomes contain transmembrane and membrane-anchored proteins that may enhance endocytosis, thus promoting the delivery of their internal content [16]. Exosomal proteins include CD47 [17], a widely expressed integrin-associated transmembrane protein that functions in part to protect cells from phagocytosis [18]. CD47 is the ligand for signal regulatory protein alpha (SIRPα, also known as CD172a), and CD47-SIRPα binding initiates the ‘don't eat me’ signal that inhibits phagocytosis.

A study from Matthew J. A. Wood's group at the University of Oxford demonstrates that exosomes stuffed full of small interfering RNA (siRNA) could reach cells inside the brains of mice [19]. Once past the brain's protective barrier, the genetic material lowered production of BACE1, a protein involved in Alzheimer's disease. Additionally, work by Raghu Kalluri's group at the University of Texas MD Anderson Cancer Center has shown the potential to overcome a problem that scientists have wrestled with for over a decade: getting siRNA into the right cells. Kalluri's team used engineered exosomes to deliver siRNA that blocked production of a mutant protein called KRas, one of the most “undruggable” cancer targets. Intravenous injections of Kalluri's siRNA-loaded exosomes suppressed pancreatic cancer in mice better than similar injections of siRNA-loaded lipid nanoparticles, and without any obvious immune reactions [20]. Kalluri is now a co-founder of Massachusetts-based Codiak Biosciences (www.codiakbio.com), one of a growing number of biotech start-ups attempting to hijack that messenger system by exosome to ferry drugs into cells in parts of the body, like the brain, that would otherwise be difficult to reach. However, isolating a large amount of the vesicles is still one of the big challenges in exosome-based therapeutics. Another challenge is how to separate and fractionate exosomes given the extracellular vesicle diversity. Consistency and reproducibility in exosome-based therapies could be compromised if all the exosomes are different sizes and thus loaded with different amounts of drugs.

In order to facilitate the diagnostic and therapeutic applications of these unique extracellular vesicles (EVs), it is crucial that exosomes are specifically isolated from a wide spectrum of cellular debris and interfering components [21]. Disease diagnostics often involve quantification of the RNA cargo within the exosomes and therapeutics require high yield harvesting of the exosomes from cell cultures. Consequently, the techniques employed in the isolation of exosomes should exhibit high efficiency and are capable of isolating exosomes from various samples. Additionally, the isolation technology should be capable of concentrating and fractionating exosomes given the diverse extracellular vesicle diversity and requirement of downstream analysis and application.

Existing commercial techniques exploit a particular trait of exosomes, such as their density, solubility, size, and surface proteins to aid their isolation: Ultracentrifugation (UC) is one of the most common techniques to separate exosomes from other EVs with different sizes and masses is ultracentrifugation, which typically requires a sequence of centrifugation steps eventually reaching speeds of up to 200000×g. This technique is time-consuming (>4 h) and provides low exosome recovery (typically <25%) [22] and low purity because of the presence of nonexosomal protein and microvesicular debris, and the equipment is relatively expensive (>$100K). Density-gradient separation is used to purify exosomes by separating them from large proteins. This technique is performed by loading the sample over a concentrated solution of the medium (sucrose or inorganic salts) and applying ultracentrifugation to extract the exosomes from other particles (proteins) based on their different flotation densities. Although density-gradient separation techniques can improve the purity and recovery rate of exosomes, they require even longer times (21 h) compared with conventional ultracentrifugation and greater technical ability of the user [23-24].

Precipitation is another common exosome isolation method. Recently, commercial rapid precipitation kits such as ExoQuick-TC and Total Exosome Isolation have been able to provide more affordable (in the range of $200-1000) approaches for many standard hospital laboratories or hospitals in resource-poor countries compared with centrifugation techniques. The proprietary polymer in these kits gently precipitates exosomes, which are then isolated by centrifugation at lower g forces, such as a typical benchtop microfuge. Although these methods avoid the issue of equipment cost, the long isolation time (˜24 h) remains a limiting factor for these techniques. The presence of a non-exosomal origin such as RNA binding proteins along with extracted exosomes is a common contaminant, preventing detection of the exosomal RNA of interest. An alternative to UC is immunoaffinity capture by magnetic beads or antibody functionalized pillars/packings and immune precipitation. The technique is limited to EVs with known antigens (CD63, CD9, CD81 of the tetraspanin family, annexin, or EpCAM). It allows isolation of EVs with these antigens from the contaminating proteins and other vesicles. However, the heterogeneity of EVs produced by cells limits the efficacy of this approach [25]. Studies have revealed that that there is no common protein that is abundantly expressed on the surface of EVs derived from diverse origins [26]. Even carriers from cancer cells may not have the cancer specific EPCAM antigens. Immunocapture hence minimizes protein and other contaminations but is generally too specific for an agnostic platform. In addition, although magnetic beads allow flow cytometry sorting and other analyses of EVs, the isolation process requires more than a day to achieve optimal recovery rates.

Size-based ultrafiltration is a commercial size-based separation technique applied to exosome isolation is size exclusion chromatography (SEC) such as IzonqEV. In SEC, a porous stationary phase is utilized to sort macromolecules and particulate matters out according to their size. Components in a sample with small hydrodynamic radii can pass through the pores, thus resulting in late elution. Components with large hydrodynamic radii including exosomes, are excluded from entering the pores. Because SEC is typically performed using gravity flow, vesicle structure and integrity largely remain intact and the biological activity of exosomes is preserved. Moreover, SEC has excellent reproducibility. However, the current manual process for isolation with qEV is not scalable which limits its scalability for high throughput applications. Another popular size-based exosome isolation technique is ultrafiltration. The fundamentals of ultrafiltration are no different from conventional membrane filtration in which the separation of suspended particles or polymers is primarily dependent on their size or molecular weight. Ultrafiltration is faster than ultracentrifugation and does not require special equipment. However, the use of force result in the deformation and breaking up of large vesicles which biases the results of downstream analysis [27-29]. Moreover, contamination by blood protein (mostly albumin) remains an issue with SEC, even with the chromatograph separation.

Although ultracentrifugation and rapid precipitation are readily available current methods to isolate exosomes, their low yield recovery and long isolation time make them unsuitable for diagnostic and therapeutic applications. Exosomes isolated by using differential ultracentrifugation often contain proteins and lipoproteins. Due to the complexity of biological samples, contamination from other extracellular vesicles with similar physicochemical and biochemical properties is unavoidable. For example, there is significant overlap in physical characteristics like density and solubility between exosome and non-exosome EVs. In contrast, size is a robust physical characteristic that is currently used to differentiate exosomes from other EVs [30]. For example, most proteins and lipoproteins have a size ranging from 2 to 35 nm while exosome are usually ranging from 50 to 200 nm. Moreover, differences in EV size has shown to influence their recognition and capture by target cells [31].

Sized-based SEC potentially allows higher purity and yield but is incapable of concentrating and fractionating exosomes. Size-based ultrafiltration is faster than ultracentrifugation and does not require special equipment. It also allows simultaneous isolation, concentration, and fractionation. However, vesicle deformation, lysing and fusion reduce the yield and potentially skew the results of downstream analysis. In addition, ultrafiltration can result in clogging and vesicle trapping, thus leading to reduce lifetime of the membranes and low isolation efficiency.

Current commercial size-based exosome isolation technologies fail to have high yield and purity, while failing to maintain the capability of concentration and fractionation. Thus, there is a need for a high-yield sized-based fractionation of isolated exosomes.

### SUMMARY

One embodiment described herein is a system for isolating exosomes comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a sample comprising exosomes positioned within the first chamber; and a device for inducing fluid flow through the membrane from the first chamber to the second chamber by pressure driven flow, electroosmotic flow, centrifugal force, or a combination thereof. In one aspect, the first chamber comprises a wall opposite of the first membrane that comprises one or more baffles.

Another embodiment described herein is a system for isolating exosomes comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; wherein the first chamber comprises a wall opposite of the first membrane that comprises one or more baffles; a sample comprising exosomes positioned within the first chamber; and a device for inducing fluid flow through the membrane from the first chamber to the second chamber by pressure driven flow, electroosmotic flow, centrifugal force, or a combination thereof. In one aspect, the first membrane surface is coated with a magnetic alloy. In another aspect, the first diameter is between about 10 nm and about 200 nm. In another aspect, the second diameter is less than about 2 μm. In another aspect, the membrane is formed from one or more materials comprising one or more of a polyethylene terephthalate (PET), a polycarbonate (PC), a polypropylene (PP), a polyimides (PI), or a polyethersulphone (PES). In another aspect, the system further comprises a third chamber and a filter positioned between the third chamber and the first chamber, the filter comprising a first filter surface facing and at least partially defining the third chamber, a second filter surface facing and at least partially defining the first chamber and a plurality of filter pores extending between the first and second filter surfaces. In another aspect, each filter pore has a diameter of 200 nm to 5 microns. In another aspect, the filter is formed from one or more materials comprising a polyethylene terephthalate (PET), a polycarbonate (PC), a polypropylene (PP), a polyimides (PI), and a polyethersulphone (PES). In another aspect, the system further comprises a fourth chamber and a second membrane positioned between the fourth chamber and the second chamber, comprising a first membrane surface facing and at least partially defining the second chamber and a second membrane surface facing and at least partially defining the fourth chamber; wherein the second membrane is the membrane as described herein; and, wherein the first membrane surface is coated with a magnetic alloy. In another aspect, the device for inducing fluid flow generates a flow rate of between about 0.01 mL/hour to about 1000 mL/hour. In another aspect, the device for inducing fluid flow generates a pressure less than about 1 atm. In another aspect, the device for inducing fluid flow comprises a syringe pump, an electroosmotic pump, a micropump, a centrifuge, or a combination thereof. In another aspect, the sample is applied perpendicularly or tangentially to the membrane or the filter. In another aspect, the magnetic alloy is nickel-iron, samarium-cobalt, aluminum-nickel-cobalt, or neodymium-iron-boron. In another aspect, the exosomes are bound to a probe that is coupled to a magnetic bead. In another aspect, the probe is an antibody.

Another embodiment described herein is a method for isolating exosomes comprising: providing a system as described herein, and inducing fluid flow through the membrane from the first chamber to the second chamber, whereupon the exosomes are isolated in the second chamber.

Another embodiment described herein is an exosome isolated using the methods described herein.

Another embodiment described herein is a method for isolating exosomes comprising: providing a system comprising: a first chamber; a second chamber; a third chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a filter positioned between the third chamber and the first chamber, the filter comprising a first filter surface facing and at least partially defining the third chamber, a second filter surface facing and at least partially defining the first chamber and a plurality of filter pores extending between the first and second filter surfaces; and a device for inducing fluid flow through the filter from the third chamber to the first chamber and through the membrane from the first chamber to the second chamber by pressure driven flow, electroosmotic flow, centrifugal force, or a combination thereof; introducing a sample comprising exosomes into the third chamber; inducing fluid flow through the filter and the membrane from the third chamber to the first chamber and from the first chamber to the second chamber, whereupon the exosomes pass through the filter and are isolated in the second chamber. In one aspect, the system further comprises a fourth chamber and a second membrane positioned between the fourth chamber and the second chamber, comprising a first membrane surface facing and at least partially defining the second chamber and a second membrane surface facing and at least partially defining the fourth chamber; wherein the second membrane is the membrane as described herein; and, wherein the first membrane surface is coated with a magnetic alloy. In another aspect, the first membrane surface is coated with a magnetic alloy. In another aspect, the sample comprising exosomes comprises one or more of cell culture supernatants, a sample obtained from an animal subject, or an apoplastic fluid from a plant. In another aspect, the sample obtained from an animal subject comprises one or more of blood, plasma, tear, serum, urine, sputum, pleural effusion, or ascites. In another aspect, the first diameter is between about 10 nm to about 200 nm. In another aspect, the second diameter is less than about 2 μm. In another aspect, the membrane is formed from one or more materials comprising a polyethylene terephthalate (PET), a polycarbonate (PC), a polypropylene (PP), a polyimides (PI), or a polyethersulphone (PES). In another aspect, each filter pore has a diameter of 200 nm to 5 microns. In another aspect, the filter is formed from one or more materials comprising a polyethylene terephthalate (PET), a polycarbonate (PC), a polypropylene (PP), a polyimides (PI), or a polyethersulphone (PES). In another aspect, the first chamber comprises a wall opposite of the first membrane that comprises one or more baffles. In another aspect, the device for flowing the sample generates a flow rate of between about 0.01 mL/hour to about 1000 mL/hour. In another aspect, the device for inducing fluid flow generates a pressure less than about 1 atm. In another aspect, the device for inducing fluid flow comprises a syringe pump, an electroosmotic pump, a micropump, a centrifuge, or a combination thereof. In another aspect, the sample is applied perpendicularly or tangentially to the filter. In another aspect, the magnetic alloy is nickel-iron, samarium-cobalt, aluminum-nickel-cobalt, or neodymium-iron-boron. In another aspect, the exosomes are bound to a probe that is coupled to a magnetic bead. In another aspect, the probe is an antibody.

Another embodiment described herein is an exosome isolated using any of the methods described herein.

### DESCRIPTION OF THE DRAWINGS

FIG. 1. Physical characteristics of the different EV subtypes. Nanoparticles that carry molecular biomarkers like proteins and RNAs: Different nanoparticles come from different cells or from different parts of the same cells. Some are only formed during acute stress. Hence, by fractionating them, one can do a deep dive on the origin of their molecular cargo. They range in size and in specific density. Many nanoparticle separation technologies are based on these differences.

FIG. 2A shows a schematic diagram of ANM fabrication process.

FIG. 2B shows a scanning electron microscope (SEM) image of the tip side before protocol optimization.

FIG. 2C shows an SEM image of the tip side after protocol optimization.

FIG. 2D shows an SEM image of both the tip and base side of 10 nm symmetric nanopore membranes, 10 nm ANMs, and 20 nm ANMs.

FIG. 3 shows the tangential-flow ANM filtration setup.

FIG. 4A and FIG. 4B are pictures of the tangential-flow ANM filtration prototype.

FIG. 4C shows the schematics of the baffle design.

FIG. 4D is a 3D printed membrane holder with the baffle design.

FIG. 5A shows the amount of isolated EVs and pressure using different membranes with different degree of pore asymmetry.

FIG. 5B shows the pore size distribution before isolation (left) and after isolation using ANM (middle) and cylindrical nanopore membranes (right).

FIG. 6A is an estimation of pressure exerted on the EVs using different isolation methods.

FIG. 6B and FIG. 6C are comparisons of the amount of isolated EVs using different isolation methods.

FIG. 6D shows an SEM image of isolated EVs and western blot analysis of exosomal marker CD63.

FIG. 7A shows schematics of exosome isolation using the tangential-flow ANM nanofiltration device.

FIG. 7B shows the protein concentration in the flow through as a function of the volume of the washing buffer pumped through the device.

FIG. 7C shows the size distribution before and after isolation.

FIG. 7D is the extraction yield comparison between ANM with other commercial techniques (ExoQuick-TC, qEV) and with UC. The use of tangential flow increases the isolation yield to 90%.

FIG. 8 shows size-based EV fractionation using 200 nm ANM (FIG. 8A) and 100 nm ANM

(FIG. 8B).

FIG. 9A is the workflow of immunocapture using Magnetic Nanopore Membrane (MNM).

FIG. 9B is a picture of the MNM made by conventional rotating stirring electroplating (left) and customized stirring device (right). FIG. 9C shows a device for running the nano-immunocapturing experiment.

FIG. 9D shows an SEM image of a NiFe layer deposited with original plating solution.

FIG. 9E shows an SEM image of a NiFe layer deposited with the saccharin-free plating solution.

FIG. 10A is a SEM image of the magnetic nanobeads (MNB) captured on the surface of the membrane.

FIG. 10B shows the recovery rates of the MNB for different conditions.

FIG. 11A shows the workflows for MNM capturing after ANM isolation and direct MNM capturing.

FIG. 11B shows the yield of MNM capturing after ANM isolation and direct MNM capturing.

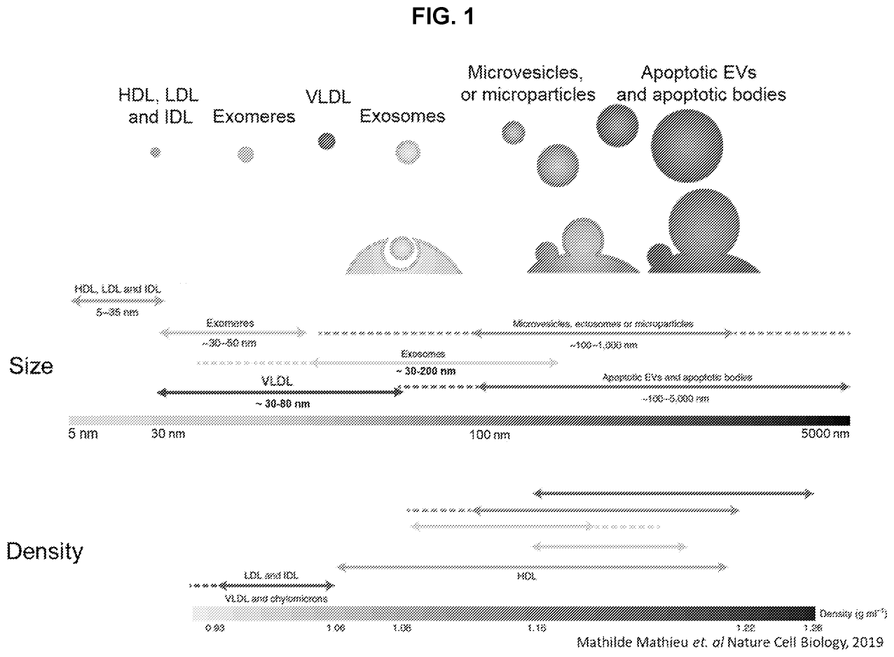

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. In case of conflict, the present document, including definitions, will control. Preferred methods and materials are described below, although methods and materials similar or equivalent to those described herein can be used in practice or testing of the present invention.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more than 3 standard deviations, per the practice in the art. Alternatively, such as with respect to biological systems or processes, the term “about” can mean within an order of magnitude, in some embodiments within 5-fold, and in some embodiments within 2-fold, of a value.

All ranges disclosed herein include both end points as discrete values as well as all integers and fractions specified within the ranges. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points. As used herein, the symbol “˜” means “about.”

The term “exosome” refers to cell-derived vesicles having a diameter of between about 20-250 nm, such as between 40 and 210 nm, for example, a diameter of about 50 nm, 60 nm, 70 nm, 80 nm, 90 nm, 100 mm, 110 nm, 120 nm, 130 nm, 150 nm, 160 nm, 170 nm, 180 nm, 190 nm, or 200 nm. Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g., immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like). As one of skill in the art will appreciate, cultured cell samples will be in the cell-appropriate culture media (using exosome-free serum). Exosomes include specific surface markers not present in other vesicles, including surface markers such as tetraspanins, e.g., CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein). Exosomes may also be obtained from a non-mammal or from cultured non-mammalian cells. As the molecular machinery involved in exosome biogenesis is believed to be evolutionarily conserved, exosomes from non-mammalian sources include surface markers which are isoforms of mammalian surface markers, such as isoforms of CD9 and CD63, which distinguish them from other cellular vesicles. The term “non-mammal” is meant to encompass, for example, exosomes from microorganisms such as bacteria, flies, worms, plants, fruit/vegetables (e.g., corn, pomegranate), and yeast.

As used herein, “sample” can mean any sample in which the presence and/or level of a target is to be detected or determined or any sample comprising an exosome, or component thereof as described herein. Samples may include liquids, solutions, emulsions, or suspensions. Samples may include any plant fluid or tissue, such as apoplastic fluid, any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, pleural effusion, ascites, digestive fluid, skin, or combinations thereof. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, the term “subject” refers to an animal. Typically, the animal is a mammal. A subject also refers to, for example, primates (e.g., humans, male or female; infant, adolescent, or adult), pigs, cows, sheep, goats, horses, dogs, cats, rabbits, rats, mice, fish, birds, and the like. In one embodiment, the subject is a human.

Described herein is a size-based asymmetric nanopore membrane (ANM) filtration technology for high-efficiency exosome isolation, concentration, and fractionation. The ANM technology utilizes an asymmetric etching technique for commercial ion-track membranes to produce conic nanopores that can range from 10 nm to 200 nm on the tip side and up to 2 microns on the base side. Track-etched membranes that have asymmetrically shaped pores (as opposed to the more conventional cylindrical or irregularly shaped pores in ultrafiltration membranes) offer an important advantage for exosome isolation applications. The key advantage of the symmetrical pore shape is a dramatic 200-400% reduction in the applied pressure/force to drive the sample through the filter membrane at the same throughput, compared to an analogous cylindrical pore membrane. This significant reduction in applied pressure prevents exosome deformation, lysing and fusion and thus significantly increases the yield (up to 90%) while preserving other advantages of size-based ultrafiltration. Moreover, the chance of clogging and vesicle trapping is significantly reduced due to a dramatic enhancement in the rate of transport through the membrane, relative to an analogous cylindrical pore membrane. This new pore geometry design allows high yield and high throughput and permits trapping designs. The trapping design allows for concentration of exosomes within a specific size range and separation from the larger and smaller debris, molecules, and EVs. The concentration factor can be as large as 100. Importantly, the trapping design allows for flushing of the trapped exosomes with rinsing buffer to remove all contaminants, including the abundant proteins. It also offers higher throughput, yield, sample purity and concentration factor than current products, plus more precise size fractionation.

The ANM is high throughput, as the conic geometry reduces the flow shear rate. The lower shear rate also minimizes nanoparticle loss due to lysing. The result is a high-yield and high-throughput platform that can isolate exosomes (about 50 to 200 nm in size) from proteins, RNPs, HDL, and LDL. The conic nanopore is fabricated by asymmetric wet etching of ion-track membranes without dielectric coating. The technology has been validated with cell media/supernatant and plasma samples. ANM exhibits much higher yield and throughput than precipitation technology (Exoquick), ultracentrifugation, size-exclusion (qEV), and column adsorption (miReasy). The throughput is particularly high, taking about 1 hour for about 1 mL cell media and about 300 microliter plasma, compared to days for the other technologies. qEV has a comparable throughput but it does not fractionate.

The isolated and purified exosomes can be lysed mechanically, thermally, or chemically to release their molecular biomarker cargo for quantification. Such quantification can be done with many technologies, including ANM miRNA quantification technology that does not suffer from PCR-amplification bias. The AMN filtration technology allows for complete EVs and protein separation due to the presence of the 30 nm asymmetric nanopore filter and the addition buffer washing step for the trapped exosomes between the two membranes. Thus, high recovery efficiency can be achieved without sacrificing protein removal. Additionally, this method doesn't require timing which introduces significant complexity in the isolation process and reduces throughput. The ANM technology isolates and concentrates EVs at the same time from any arbitrary volume up to 5 mL, up to 4 mL, up to 3 mL, up to 2 mL, up to 1mL, up to 500 μL, or up to 300 μL. The concentration factor can be as large as a factor of 10 to 100. The present nanopore technology allows the same isolation efficiency for all exosomes with a size larger than the tip size of the pore, thus less bias is introduced in the isolation step. AMN technology allows for precise control of the pore size such that size-based fractionation can be performed within the 30-200 nm range (by using different nanopore membrane modules with different pore sizes).

ANM consists of a membrane holder and a commercial micropump or syringe pump. The pump can be housed in a dedicated instrument or the consumers can use their own syringe pumps in their laboratories. One embodiment includes the ANM and its holder, which may be disposed after each use. The ANM may be fabricated from the polycarbonate track-etched membranes, which are initially irradiated to create the desired ion tracks and then etched to develop tracks into pores. The track irradiation step is capable of mass production. The etching process involves chemical etching and dry etching, which are also easy to scale up.

One embodiment described herein is a system for isolating exosomes comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a sample comprising exosomes positioned within the first chamber; and a device for inducing fluid flow through the membrane from the first chamber to the second chamber by pressure driven flow, electroosmotic flow, centrifugal force, or a combination thereof. In one aspect, the first chamber comprises a wall opposite of the first membrane that comprises one or more baffles. In some embodiments, there may be at least 1, at least 2, at least 3, at least 4, at least 5 baffles. In other embodiments, there may be at most 1000, at most 900, at most 800, at most 700, at most 600, at most 500, at most 400, at most 300, at most 200, at most 100, at most 50, or at most 25 baffles. The baffles may be made of fiberglass, plastic, a composite, or another material. In some embodiments, the baffles may be made of polycarbonate (PC), polystyrene (PS), polyethylene terephthalate (PET), polyvinylchloride (PVC), SU-8 photoresist and polyimide (PI), polydimethylsiloxane (PDMS), silicon, or glass. In a particular embodiment, the baffles may be made of polymethyl methacrylate (PMMA). The baffles can be shaped like cubes, triangular prisms, rectangles, cones, or panels that are curved, zigzagged, corrugated or L-shaped, have a combination of these shapes, or are otherwise configured. The baffle geometry can be triangle, wedge, crescent etc. They can assume regimented or staggered patterns, including herringbone patterns. In a particular embodiment, the baffles may be cubes or triangular prisms. The baffles can have a height ranging from about 15 μm to about 3 mm, about 20 μm to about 2 mm, about 25 μm to about 2 mm, about 30 μm to about 2 mm, about 35 μm to about 1 mm, about 40 μm to about 1 mm, or about 45 μm to about 1 mm. The baffles may be spaced from about 25 μm to about 7 mm, about 50 μm to about 6 mm, about 100 μm to about 5 mm, about 100 μm to about 4 mm, about 100 μm to about 3 mm, about 100 μm to about 2 mm, about 100 μm to about 1 mm, about 125 μm to about 5 mm, or about 150 μm to about 5 mm apart. The size, number, and spacing of the baffles may vary and be selected to provide the sample flow dispersion, route, and rate desired for a particular use or particle to be isolated. In some embodiments, each or particular baffles have gaps formed at both the top and/or the bottom, at one or both sides, all the way around them. In addition, the baffles may be arranged in an array with a regular pattern or an irregular arrangement. And some of the baffles may be larger than other ones. Previously ultrafiltration baffles have been placed directly on a membrane to produce vortices that break up filter cakes. The vortices, however, will also reduce the filtration rate. The present disclosure places the baffles on the channel surface opposite of the membrane without producing vortices. The arrangement and spacing of the baffles depends on various factors such as the size range of the nanoparticles, diffusivity in that particular medium, membrane thickness, etc. and can be dictated through the diffusion timescale of the polarized layer, the normal and tangential flow rates, and the entrance length of the fluid flow. The baffles produce an upward lift to disrupt the filter cake before it is well packed.

Another embodiment described herein is a system for isolating exosomes comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; wherein the first chamber comprises a wall opposite of the first membrane that comprises one or more baffles;

a sample comprising exosomes positioned within the first chamber; and a device for inducing fluid flow through the membrane from the first chamber to the second chamber by pressure driven flow, electroosmotic flow, centrifugal force, or a combination thereof. In one aspect, the first membrane surface may be coated with a magnetic alloy. In another aspect, the system may further comprise a fourth chamber and a second membrane positioned between the fourth chamber and the second chamber, comprising a first membrane surface facing and at least partially defining the second chamber and a second membrane surface facing and at least partially defining the fourth chamber. The second membrane may be the membrane as described herein (e.g., ANM) and the first membrane surface of the membrane may be coated with a magnetic alloy. In another aspect, the magnetic alloy is nickel-iron, samarium-cobalt, aluminum-nickel-cobalt, nickel-iron-chromium, iron-chromium-cobalt, or neodymium-iron-boron. In another aspect, the exosomes are bound to a probe that is coupled to a magnetic bead. The magnetic bead may be any of a wide variety of shapes, such as spherical, generally spherical, egg shaped, disc shaped, cubical and other three-dimensional shapes. The magnetic beads may be manufactured using a wide variety of materials, including for example, resins, and polymers. The magnetic beads may be any suitable size, including for example, microbeads, microparticles, nanobeads and nanoparticles. The magnetic beads may comprise a magnetically responsive material that may constitute substantially all of a bead or one component only of a bead. The remainder of the bead may include, among other things, polymeric material, coatings, and moieties which permit attachment of an assay reagent. Examples of suitable magnetic beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Dynal Bead Based Separations (Invitrogen Group), Carlsbad, Calif.), fluorescent microparticles and nanoparticles, coated magnetic microparticles and nanoparticles, ferromagnetic microparticles and nanoparticles, coated ferromagnetic microparticles and nanoparticles, as well as other magnetic beads known in the art. In another aspect, the probe is an antibody. The antibody may bind to surface markers on exosomes. In particular, the antibody may bind to CD9, CD31, CD37, CD44, CD53, CD63, CD81, CD82 and CD151, integrins, ICAM-1, EpCAM, annexins, TSG101, ALIX, Rab5b, HLA-G, HSP70, LAMP2, LIMP, other known exosome surface markers, or a combination thereof. In another aspect, the first diameter may be between about 5 nm and about 300 nm, about 5 nm and about 200 nm, about 10 nm and about 300 nm, about 10 nm and about 200 nm, about 10 nm and about 150 nm, about 10 nm and about 100 nm, about 10 nm and about 50 nm, about 20 nm and about 300 nm, about 20 nm and about 200 nm, about 20 nm and about 100 nm, or about 50 nm and about 200 nm. In a particular aspect, the first diameter may be between about 10 nm and about 200 nm. The second diameter may be less than about 5 μm, less than about 4 μm, less than about 3 μm, less than about 2 μm, less than about 1 μm, or less than about 0.5 μm. In a particular aspect, the second diameter may be less than about 2 μm. The nanopores may be arranged in an array with a regular pattern or an irregular arrangement. And some of the baffles may be larger than other ones. In another aspect, the membrane is formed from one or more materials comprising one or more of a polyethylene terephthalate (PET), a polycarbonate (PC), a polypropylene (PP), a polyimides (PI), or a polyethersulphone (PES). In another aspect, the system further comprises a third chamber and a filter positioned between the third chamber and the first chamber, the filter comprising a first filter surface facing and at least partially defining the third chamber, a second filter surface facing and at least partially defining the first chamber and a plurality of filter pores extending between the first and second filter surfaces. In another aspect, each filter pore may have a diameter of 150 nm to 6 microns, 150 nm to 5 microns, 200 nm to 5 microns, 200 nm to 4 microns, 200 nm to 3 microns, 200 nm to 2 microns, 200 nm to 1 micron, 300 nm to 5 microns, 400 nm to 5 microns, 500 nm to 5 microns, 600 nm to 5 microns, 700 nm to 5 microns, 800 nm to 5 microns, 900 nm to 5 microns, or 1000 nm to 5 microns. In another aspect, the filter is formed from one or more materials comprising a polyethylene terephthalate (PET), a polycarbonate (PC), a polypropylene (PP), a polyimides (PI), and a polyethersulphone (PES). In another aspect, the device for inducing fluid flow generates a flow rate of between about 0.01 mL/hour to about 1000 mL/hour, about 0.01 mL/hour to about 900 mL/hour, about 0.01 mL/hour to about 800 mL/hour, about 0.01 mL/hour to about 700 mL/hour, about 0.01 mL/hour to about 600 mL/hour, about 0.01 mL/hour to about 500 mL/hour, about 0.01 mL/hour to about 400 mL/hour, about 0.01 mL/hour to about 300 mL/hour, about 0.01 mL/hour to about 200 mL/hour, about 0.01 mL/hour to about 100 mL/hour, about 0.05 mL/hour to about 1000 mL/hour, about 0.1 mL/hour to about 1000 mL/hour, about 0.2 mL/hour to about 1000 mL/hour, about 0.3 mL/hour to about 1000 mL/hour, about 0.4 mL/hour to about 1000 mL/hour, or about 0.5 mL/hour to about 1000 mL/hour. In another aspect, the device for inducing fluid flow generates a pressure less than about 0.3 atm, less than about 0.4 atm, less than about 0.5 atm, less than about 1 atm, less than about 1.1 atm, less than about 1.2 atm, less than about 1.3 atm, less than about 1.4 atm, less than about 1.5 atm. In particular, the device for inducing fluid flow generates a pressure less than about 1 atm. In another aspect, the device for inducing fluid flow comprises a syringe pump, an electroosmotic pump, a micropump, a centrifuge, or a combination thereof. In another aspect, the sample is applied perpendicularly or tangentially to the membrane or the filter.

Another embodiment described herein is a method for isolating exosomes comprising: providing a system as described herein, and inducing fluid flow through the membrane from the first chamber to the second chamber, whereupon the exosomes are isolated in the second chamber.

Another embodiment described herein is an exosome isolated using the methods described herein.

Another embodiment described herein is a method for isolating exosomes comprising: providing a system comprising: a first chamber; a second chamber; a third chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a filter positioned between the third chamber and the first chamber, the filter comprising a first filter surface facing and at least partially defining the third chamber, a second filter surface facing and at least partially defining the first chamber and a plurality of filter pores extending between the first and second filter surfaces; and a device for inducing fluid flow through the filter from the third chamber to the first chamber and through the membrane from the first chamber to the second chamber by pressure driven flow, electroosmotic flow, centrifugal force, or a combination thereof; introducing a sample comprising exosomes into the third chamber; inducing fluid flow through the filter and the membrane from the third chamber to the first chamber and from the first chamber to the second chamber, whereupon the exosomes pass through the filter and are isolated in the second chamber. In one aspect, the sample comprising exosomes comprises one or more of cell culture supernatants, a sample obtained from an animal subject, or an apoplastic fluid from a plant. In another aspect, the sample obtained from an animal subject comprises one or more of blood, plasma, tear, serum, urine, sputum, pleural effusion, or ascites. In another aspect, the first diameter is between about 10 nm to about 200 nm. In another aspect, the second diameter is less than about 2 μm. In another aspect, the membrane is formed from one or more materials comprising a polyethylene terephthalate (PET), a polycarbonate (PC), a polypropylene (PP), a polyimides (PI), or a polyethersulphone (PES). In another aspect, each filter pore has a diameter of 200 nm to 5 microns. In another aspect, the filter is formed from one or more materials comprising a polyethylene terephthalate (PET), a polycarbonate (PC), a polypropylene (PP), a polyimides (PI), or a polyethersulphone (PES). In another aspect, the first chamber comprises a wall opposite of the first membrane that comprises one or more baffles. In another aspect, the device for flowing the sample generates a flow rate of between about 0.01 mL/hour to about 1000 mL/hour. In another aspect, the device for inducing fluid flow generates a pressure less than about 1 atm. In another aspect, the device for inducing fluid flow comprises a syringe pump, an electroosmotic pump, a micropump, a centrifuge, or a combination thereof. In another aspect, the sample is applied perpendicularly or tangentially to the filter.

Another embodiment described herein is an exosome isolated using any of the methods described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The compositions, formulations, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

### REFERENCES

1. Raposo and Stoorvogel, “Extracellular vesicles: exosomes, microvesicles, and friends,” J. Cell Biol. 200(4): 373-383 (2013).
2. Pisitkun et al., “Identification and proteomic profiling of exosomes in human urine,” Proc. Natl. Acad. Sci. USA 101(36): 13368-13373 (2004).
3. Valadi et al., “Exosome-mediated transfer of mRNAs and microRNAs is a novel mechanism of genetic exchange between cells,” Nat. Cell Biol. 9(6): 654-659 (2007).
4. Mathivanan et al., “Exosomes: extracellular organelles important in intercellular communication,” J. Proteomics 73(10): 1907-1920 (2010).
5. Turchinovich et al., “Characterization of extracellular circulating microRNA,” Nucl. Acids Res. 39(16): 7223-7233 (2011).
6. Simons and Raposo, “Exosomes—vesicular carriers for intercellular communication,” Curr. Opin. Cell Biol. 21(4): 575-81 (2009).
7. Richards et al., “Cancer-associated fibroblast exosomes regulate survival and proliferation of pancreatic cancer cells,” Oncogene 36(13): 1770-1778 (2016).
8. Peinado et al., “Melanoma exosomes educate bone marrow progenitor cells toward a pro-metastatic phenotype through MET,” Nat. Med. 18(6): 883-889 (2012).
9. Raposo et al., “B lymphocytes secrete antigen-presenting vesicles,” J. Exp. Med. 183(3): 1161-1172 (1996).
10. Taylor and Gercel-Taylor, “MicroRNA signatures of tumor-derived exosomes as diagnostic biomarkers of ovarian cancer,” Gynecol. Oncol. 110(1): 13-21 (2008).
11. Gross et al., “Active Wnt proteins are secreted on exosomes,” Nat. Cell Biol. 14(10): 1036-1045 (2012).
12. Chavez-Munoz et al., “Primary human keratinocytes externalize stratifin protein via exosomes,” J. Cell. Biochem. 104(6): 2165-2173 (2008).
13. Sheridan “Exosome cancer diagnostic reaches market,” Nat. Biotechnol. 34(4): 359-360 (2016).
14. Melo et al., “Glypican-1 identifies cancer exosomes and detects early pancreatic cancer,” Nature 523(7559): 177-182 (2015).
15. Ko et al., “Detection and isolation of circulating exosomes and microvesicles for cancer monitoring and diagnostics using micro-/nano-based devices,” Analyst 141(2): 450-460 (2016).
16. Johnsen et al., “A comprehensive overview of exosomes as drug delivery vehicles-endogenous nanocarriers for targeted cancer therapy,” Biochim. Biophys. Acta 1846(1): 75-87 (2014).
17. Kaur et al., “CD47-dependent immunomodulatory and angiogenic activities of extracellular vesicles produced by T cells,” Matrix Biology 37: 49-59 (2014).
18. Jaiswal et al., “CD47 is upregulated on circulating hematopoietic stem cells and leukemia cells to avoid phagocytosis,” Cell 138(2): 271-285 (2009).
19. Alvarez-Erviti et al., “Delivery of siRNA to the mouse brain by systemic injection of targeted exosomes,” Nat. Biotechnol. 29(4): 341-345 (2011).
20. Kamerkar et al., “Exosomes facilitate therapeutic targeting of oncogenic KRAS in pancreatic cancer,” Nature 546(7659): 498-503 (2017).
21. Li et al., “Progress in Exosome Isolation Techniques,” Theranostics 7(3): 789-804 (2017)
22. Lamparski et al., “Production and characterization of clinical grade exosomes derived from dendritic cells,” J. lmmunol. Methods 270(2): 211-226 (2002)
23. Tauro et al., “Comparison of ultracentrifugation, density gradient separation, and immunoaffinity capture methods for isolating human colon cancer cell line LIM1863-derived exosomes,” Methods 56(2): 293-304 (2012)
24. Van Deun et al., “The impact of disparate isolation methods for extracellular vesicles on downstream RNA profiling,” J. Extracell. Vesicles 3(1): 248585; doi: 10.3402/jev.v3.24858 (2014).
25. Zhang et al., “Identification of distinct nanoparticles and subsets of extracellular vesicles by asymmetric-flow field-flow fractionation,” Nat. Cell Biol. 20(3): 332-343 (2018).
26. Willms et al., “Cells release subpopulations of exosomes with distinct molecular and biological properties,” Nat. Sci. Rep. 6: 22519, doi: 10.1038/srep22519 (2016).
27. Davies et al., “Microfluidic filtration system to isolate extracellular vesicles from blood,” Lab Chip 12(24): 5202-5210 (2012).
28. Liu et al., “The Exosome Total Isolation Chip,” ACS Nano 11(11): 10712-10723 (2017).
29. Woo et al., “Exodisc for Rapid, Size-Selective, and Efficient Isolation and Analysis of Nanoscale Extracellular Vesicles from Biological Samples,” ACS Nano 11(2): 1360-1370 (2017).
30. Mathieu et al., “Specificities of secretion and uptake of exosomes and other extracellular vesicles for cell-to-cell communication,” Nat. Cell. Biol. 21(1):9-17 (2019).
31. Mulcahy et al., “Routes and mechanisms of extracellular vesicle uptake,” J. Extracell. Vesicles 3(1): 24641; doi: 10.3402/jev.v3.24641 (2014)

### EXAMPLES

### Example 1

### General Methods

### Asymmetric Nanopore Membrane (ANM)

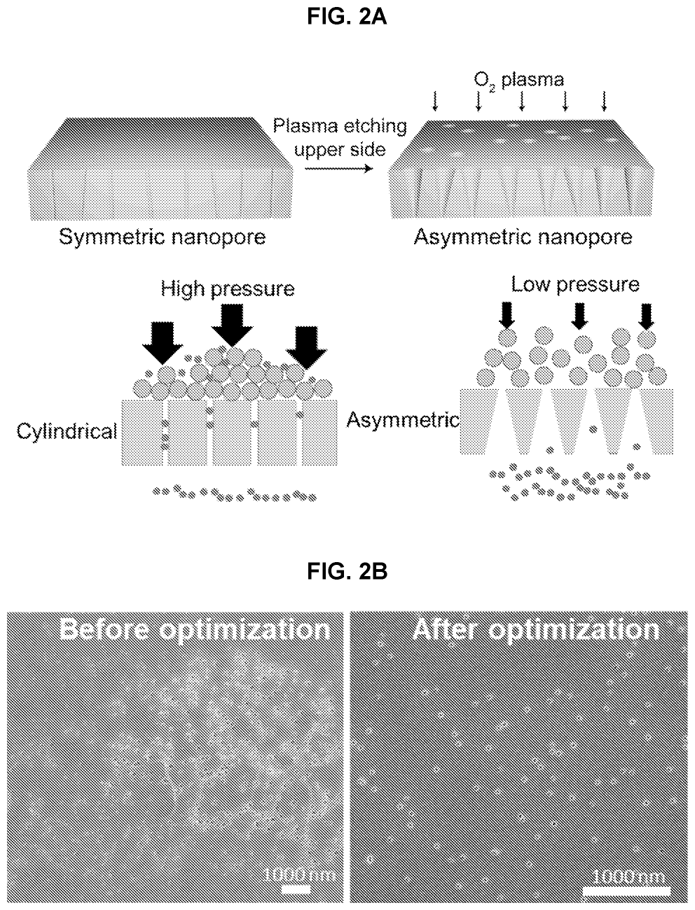

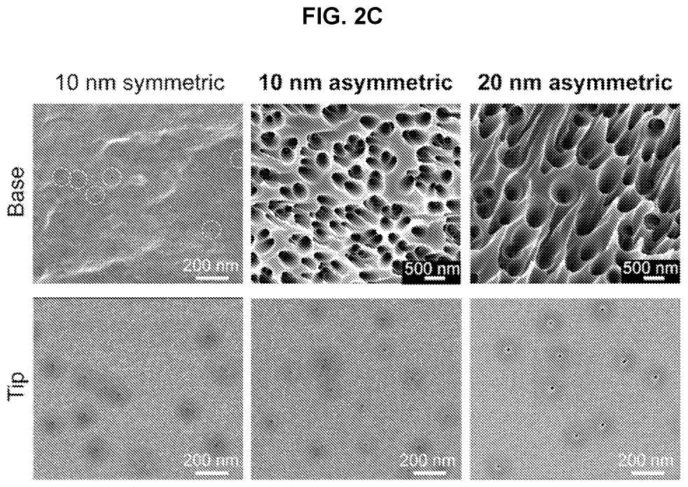

The track-etched membranes are prepared by the track-etching technique, which is based on the irradiation of a material with swift heavy ions and subsequent chemical etching. The pore size can be controlled by the etching time, and the number of ions per unit area determines the number of damage tracks and, hence, pores. Polycarbonate membranes of this type having cylindrical pores with diameters ranging from as small as 10 nm to as large as 20 μm, and pore densities as high as 5×10⁸ cm⁻², are sold commercially. 30 nm PC membranes were used in this study and were 6-μm-thick and obtained from Sigma (Whatman Nuclepore Track-Etched Membranes). The as-received membranes have a cylindrical pore shape and have a pore density of 5×10⁸ cm⁻². The pore size and density of the as-received membranes have been confirmed by SEM (FIG. 2C). Asymmetric nanopores were produced by a simple O₂ plasma etching process on one face of the as-received tracked membrane (FIG. 2A). A 25 mm-in-diameter cylindrical pore membrane was placed on a silicon wafer (500 μm thick). One surface of these membranes appears shiny and the opposite surface appears rough to the eye. The membrane was placed on the silicon wafer with the rough surface up. A 2.5 cm×2.5 cm PMMA sheet that had a 21 mm-in-diameter hole cut through it was placed on top of the membrane, and Kapton tape was used to attach the PMMA sheet to the silicon wafer. This hole defined the area of the membrane exposed to the O₂ plasma. O₂ plasma etching was performed with a commercial reactive ion etch system (Oxford PlasmaPro System, model RIE100). The etching conditions were as follows: O₂ gas pressure 200 Pa, gas flow rate 30 standard cm³ min⁻¹, and power 100 W. As indicated in FIG. 2A and FIG. 2C, plasma etching enlarges the pore diameter at the upper surface, but the pore diameter remains unchanged at the lower surface. Furthermore, plasma etching also reduces the thickness of the membrane.

### Preparation of Biofluid Samples

Whole blood samples were obtained from healthy patients and mice during fasting. For plasma samples, 2 mL of whole blood was collected in a Vacutainer tube containing EDTA as anticoagulant and centrifuged for 10 minutes at 1900×g (3000 rpm) and 4° C. to separate the plasma fraction. Fresh plasma samples (50-300 μL) were used immediately for exosome isolation experiment. The individual tissue cultures of different cell lines (LOX melanoma cell, PC3 mouse prostate cancer cell, MCF-7 breast cancer cell, OVCAR5 ovarian cancer cell) were grown in 37° C. until 90% confluent, the cell culture supernatants were harvested and centrifuged for 20 minutes at 2000×g.

### Exosome Isolation

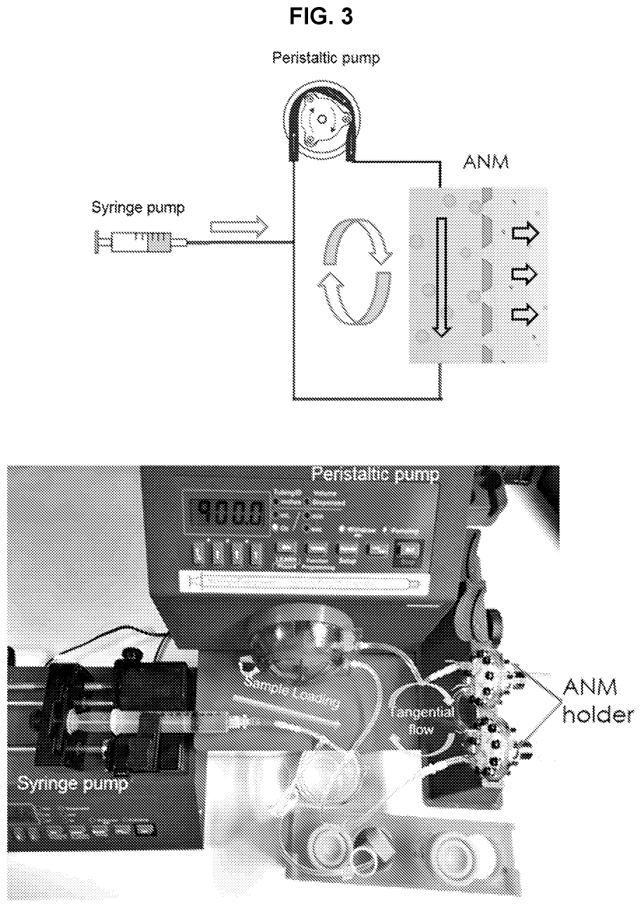

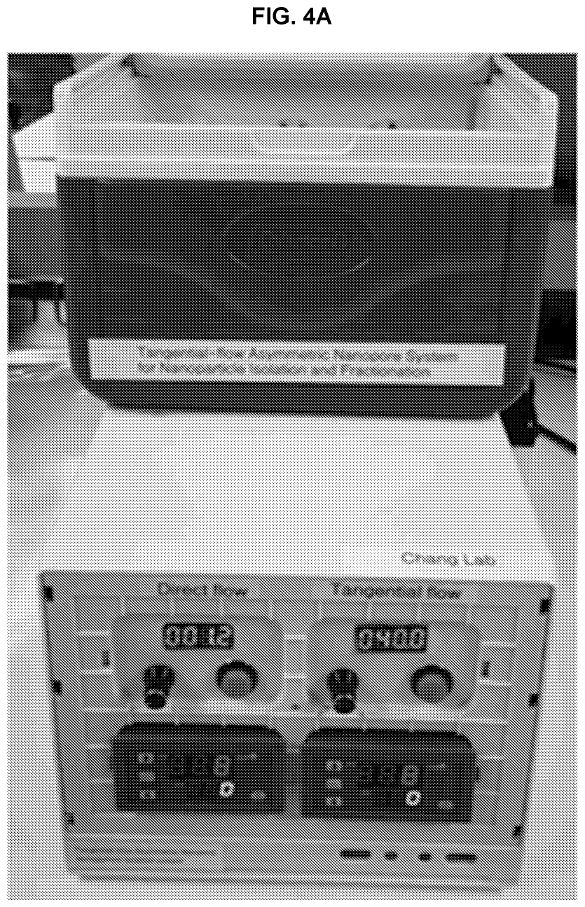

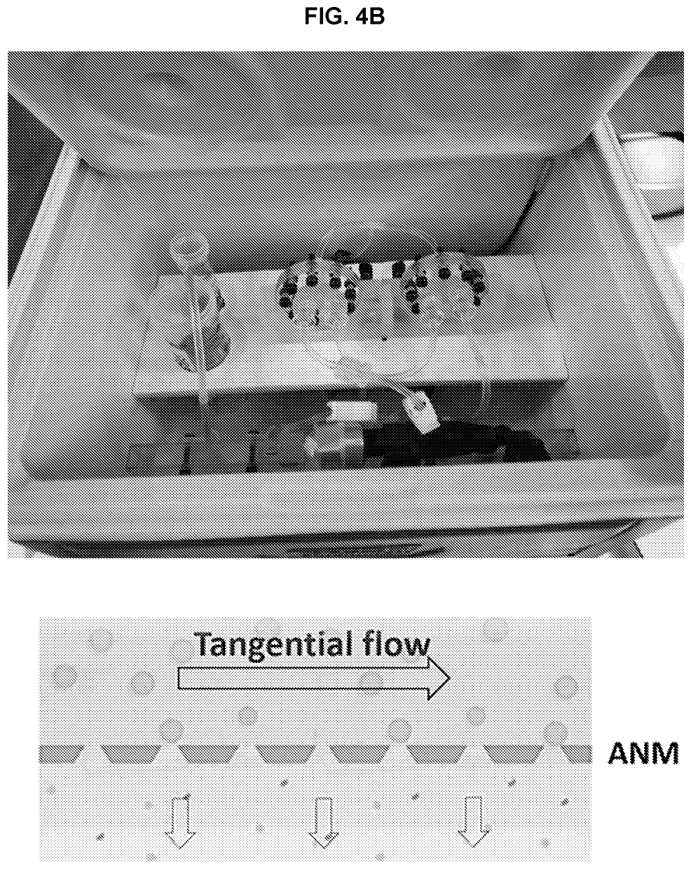

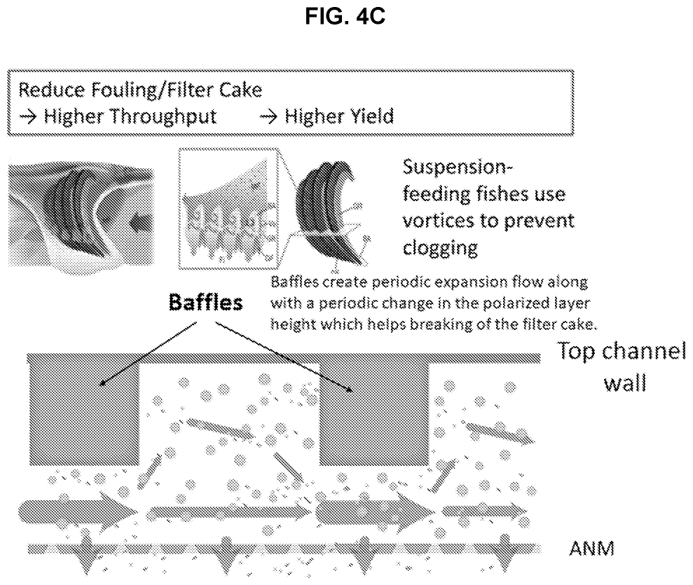

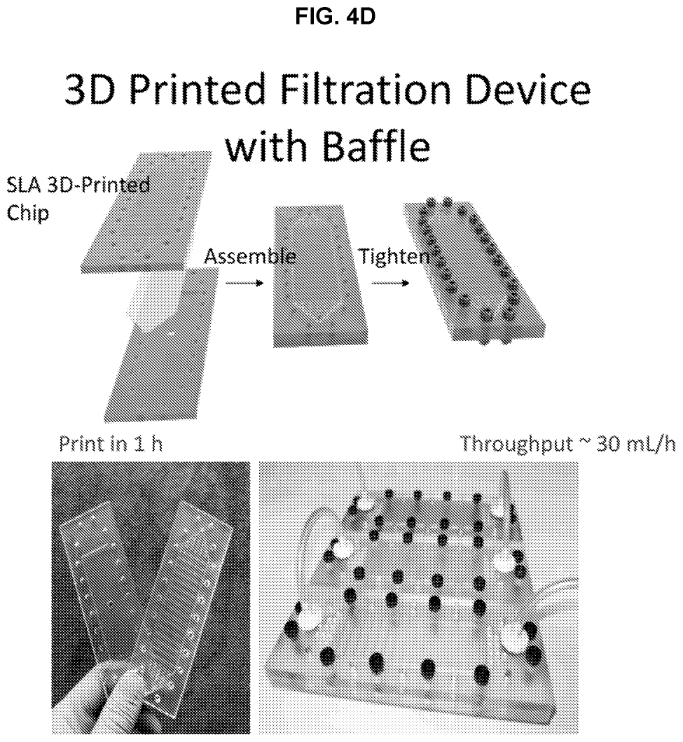

Exosome isolation was performed by direct flow nanofiltration using the as-prepared asymmetric nanopore membranes. The membrane was sealed in a home-made plastic membrane holder. The plastic housing was secured with metal screws and nuts, and a plastic ring-shaped gasket provided a leak-free seal. The isolation involved size-based isolation and washing steps. The cell culture supernatant and diluted plasma samples (dilution factor: 40) were prefiltered with a 0.22 μm PES syringe filter, and were introduced continuously into the asymmetric nanopore membrane filtration device via a 5 mL syringe using a syringe pump at a constant flow rate (5 mL/h), followed by a 5 mL 1× PBS washing step. The concentrated exosomes were recovered from the fluid chamber (volume ˜300 μL) next to the asymmetric nanopore membrane, and the isolated EVs were then used for downstream physical characterization. Exosome isolation was also performed in a tangential-flow nanofiltration mode when large-volume and heterogeneous samples were processed. Filter-cake formation and high build-up pressure lead to exosome lysing and coalescence especially when the highly heterogeneous samples are filtered in large volume. In the tangential-flow nanofiltration assay, the feed stream passes parallel to the asymmetric nanopore membrane face as one portion passes through the membrane (permeate) while the remainder (retentate) is recirculated. The tangential-flow ANM filtration platform is shown in FIG. 3. The cell culture supernatant and diluted plasma samples (dilution factor: 40) were prefiltered with a 0.22 μm PES syringe filter, and were introduced continuously by the syringe pump at a flow rate (10 mL/h) while the peristaltic pump recirculates the retentate stream at a flow rate (40 mL/h) to prevent the formation of a restrictive layer, followed by a wash step comprising up to 30 mL 1×PBS. The ANM flow chip was made by 3D printing the chip with a channel dimension of 65 (L)×20 (W)×1 (H) mm. A baffled tangential flow design was also introduced to better suppress the fouling and filter cake formation. These baffles were fabricated on the top wall of the flow channel such that the baffles are part of the flow chamber which is made of polymethyl methacrylate (PMMA) as shown in FIG. 4D. The baffles can be shaped like cubes or triangular prisms. The baffles can have a height ranging from about 25 μm to about 2 mm and be spaced from about 100 μm to about 5 mm apart. The baffle design allowed for a different shear rate and polarized layer length of the filter cake at the baffle and the spacing between the baffle as shown in FIG. 4C. The difference in characteristic polarized length and shear rate of the filter cake allows it to break at the point of change. A two-dimensional baffle can also induce vortices in the system that breakup the filter cake. The baffle design was inspired by a specialized filtering structure in filter feeder (e.g., suspension feeding) fish, the specialized filtering structure can significantly enhance the restrictive clogging layer removal by inducing localized vortices as illustrated in FIG. 3. The concentrated exosomes were recovered from the flow chip (volume ˜2 mL) next to the asymmetric nanopore membrane.

### MNM Fabrication

Briefly, 80 nm Au was deposited using a thermal evaporator (Oerlikon Leybold 8-pocket electron-beam) onto one side of a 450 nm track-etched polycarbonate membrane (Whatman) to provide a working electrode in the subsequent electrodeposition process. Then 200 nm Ni₈₀Fe₂₀ film was electrodeposited on top of the Au film. An Ni electrode was used in the electrodeposition solution. Ni₈₀Fe₂₀ electrodeposition solution was composed of 289 g/L NiSO₄.6H₂O, 64 g/L FeSO₄,7H₂O, 40 g/L H₃BO₃, 8.9 g/L 5-Sulfosalicylic acid dihyfrate, and 3 g/L 1,3,(6,7)-Naphthalenetrisulfonic acid trisodium salt hydrate. During the electrodeposition, the deposition current <2.5 mA/cm². The resulting MNM has an asymmetric geometry with a base diameter of about 450 nm and a tip diameter of about 250 nm.

### Exosome Isolation Using MNM

Exosomes were first isolated based on their size using ANM from mouse plasma, as detailed herein. Immuno-sorting of exosomes is performed by positive selection using magnetic nanobeads recognizing the tetraspanin proteins CD9, CD63, or CD81 (Miltenyi Biotec Inc.). These magnetic nanobeads (20-30 nm) with antibodies were added to the sample (isolated exosomes) and incubated for 30 min at room temperature with shaking. Then the samples were added to the reservoir of the MNM holder and pressure was applied by a programmable syringe pump to pump the exosome sample at a flow rate of 1 mL/h. The MNM holder was fabricated by a computer-controlled milling machine (Roland, monoFab SRM-20). Two ring neodymium magnets were placed on the top and bottom side of the MNM holder, respectively, which provide the magnetic field to magnetize the MNM. As the sample solution was pumped through the chip, exosomes that were labeled with magnetic nanoparticles were captured at the edge of the pores of the MNM.

### Characterization of Isolated Exosomes

The isolated exosome samples collected from the chips were then diluted in 1×PBS buffer ˜20-500-fold. Nanoparticle tracking analysis was performed using a Nanosight NS300 (NanoSight, Wiltshire, UK). Five 60-second videos were recorded of each sample with camera level and detection threshold set at 10.

### Example 2

### ANM Fabrication

Ion-track membranes have uniform pore dimensions and straight pores. They are better than commercial ultrafiltration membranes (e.g., PES membranes), that have irregular pore geometry and non-specific binding, for EV nanofiltration. However, ion-track membranes have two primary disadvantages: (1) fusion/clogging in the nanopore and (2) filter cake formation when the membrane surface is not sufficiently large for concentrated suspensions like EV in plasma. The conic pore geometry of the present disclosure prevents fusion and clogging of the nanopore. In addition, the conic pore geometry allows for mild operating force, has low resistance to the flow of a sample, and has reduced fouling. It is based on reactive ion etching (RIE) of commercial ion track membranes to produce ANMs with uniform tip size and pore geometry, as shown in FIG. 2A-FIG. 2C. After calibrating the etching rate and optimizing all the parameters (such as the residual stress of an unetched nanopore membrane, the resistance of the substrate wafer, and the spacing between the unetched membrane and the substrate) this novel etching protocol resulted in a greater than 90% success rate for obtaining uniform ANMs with reproducible filtration performance. To fractionate EVs based on size, different ANMs with different cutoff pore sizes were developed. The novel fabrication protocol is capable of fabricating ANMs with a tip pore size ranging from about 10 nm to about 200 nm. FIG. 2C shows representative Scanning Electron Microscopy (SEM) images of 10 nm and 20 nm ANMs. The precise cutoff pore size control (down to 10 nm) of ANMs may enable direct fractionation of ultra-small extracellular RNA carriers such as high-density lipoproteins (HDLs) and exomeres.

### Example 3

### Design of Tangential-Flow ANM Nano-Filtration Platform

A tangential-flow ANM nano-filtration design was implemented to overcome filter cake formation. Two designs were developed: (1) a “dead-end” filtration cassette, where the feed stream is applied perpendicular to the membrane face and passes 100% of the fluid through the membrane, and (2) a tangential-flow filtration chip, where the feed stream is applied parallel to the membrane face and one portion of the feed stream passes through the membrane (permeate) while the remainder (retentate) may be recirculated or fed into to the next chip. A recirculating flow may also be used with the dead-end design. To minimize filter cake formation, the membrane area was increased by connecting different cassettes or chips. Baffles were added on the top substrate of the tangential-flow filtration chip. The channel height within the 3-D printed tangential-flow chip was optimized to 1 mm to reduce dead volume and yet allow high throughput (FIG. 3). Flow-directing pillars were fabricated at the inlet and outlet of the channel to distribute flow evenly. A portable instrument for the tangential-flow ANM filtration chip was developed as shown in FIG. 4A. A three-stage design shown in FIG. 4D enabled a significantly enhanced throughput −30 mL/h for cultured medium samples and 1 mL plasma/h for plasma samples, after 30× dilution.

### Example 4

### Validation of ANM Technology

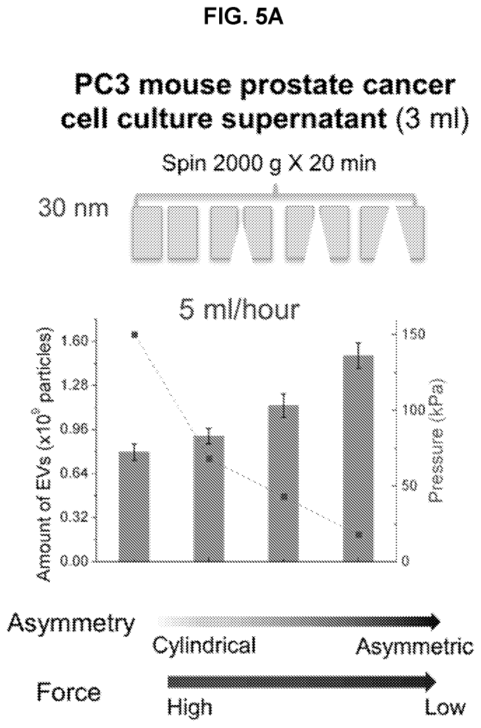

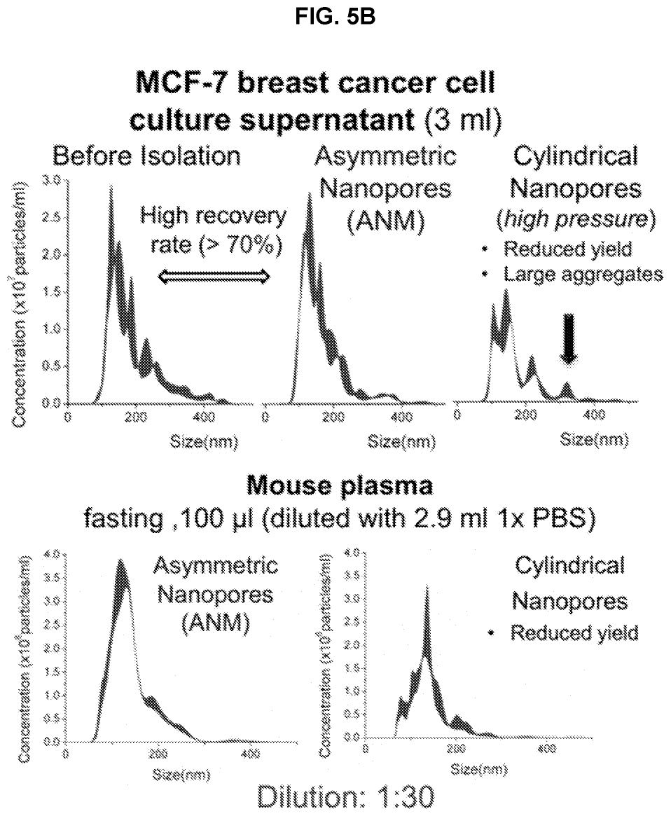

To highlight the unique advantage of ANM filtration, a study was performed to demonstrate how the conic pore asymmetry of the ANM significantly reduces the pumping pressure as well as clogging. By stopping the etching process at different time intervals, the pore geometry was tuned from a cylinder to a perfect cone (FIG. 5A). The pumping pressure drops significantly when the cone is fully formed. For the straight pore membrane, the pressure was as high as ˜150 kPa while the pressure was only 20 kPa for the conical nanopore membrane—the pressure was reduced by a factor of 7.5 for the ANM. At the same time, Nanoparticle Tracking Analysis (NTA) indicated that the numbers of isolated EVs were doubled when conic geometry was used. A close look at the particle size distribution (FIG. 5B) in the retentate of the straight pore membrane revealed significant EV loss and appearance of larger particles formed by fusion (represented by the new peaks beyond 200 nm). For the ANM, such size-distribution distortion was absent and the power-law size distributions of EVs were similar before and after filtration. Hence, ANM enables a high yield of larger than 70%, which was consistent and observed for different cell lines as well as plasma samples. This shows that the ANM filtration technology is robust and can be used for various cell and sample types. Further, these results indicate that fusion causes the majority of nanocarrier loss and that the conic pore geometry of the ANM minimizes this loss. Since the conic geometry also reduces the pressure gradient, a correlation between pressure and loss has been established.

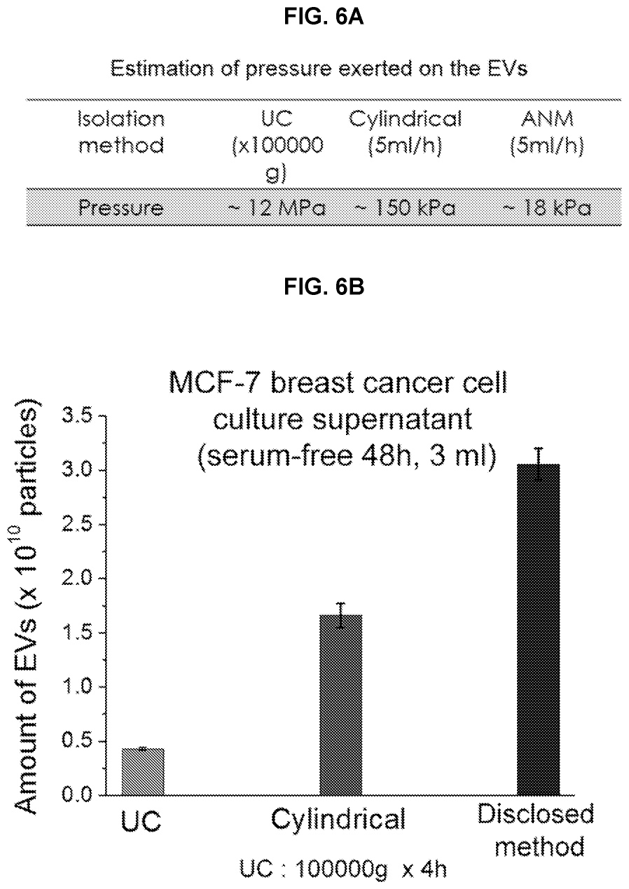

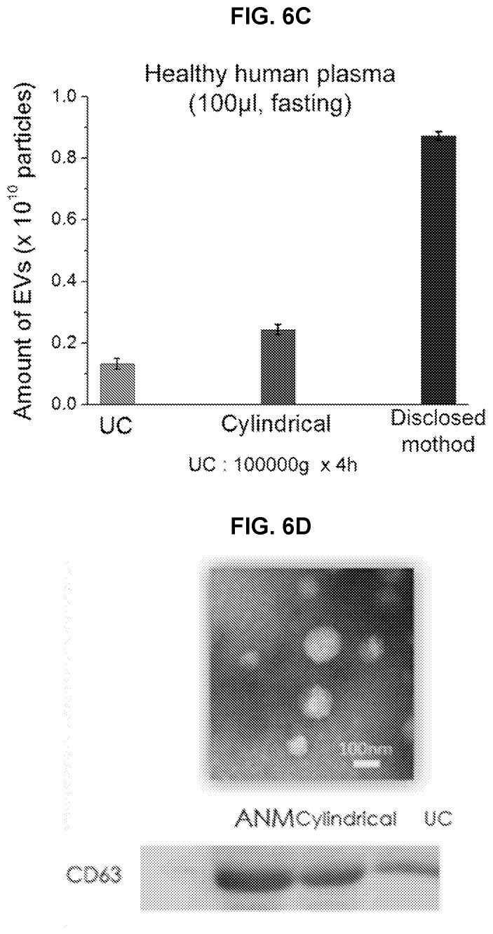

The ANM filtration technology was compared against other commonly used EV isolation technologies. FIG. 6A and FIG. 6B show an estimation of pressure exerted on EVs and a yield comparison of isolated EVs using different isolation strategies, respectively. The ultracentrifugation (UC) method utilized a high g-force (˜100000 g) to separate the EVs, which can be converted to an equivalent inertial pressure of 120 atm. In comparison, the straight pore membrane and the ANM with a flow rate of 5 mL/h required a much lower pressure of about 1.5 atm and 0.2 atm, respectively. As shown in FIG. 6B, the UC method had the lowest yield while the ANM isolated the highest number of EVs. This demonstrates that there is a negative correlation between yield and pressure. These results were also confirmed with the expression levels of isolated exosomal protein biomarker (CD63) by western blot analysis (FIG. 6D). The high yield of EV isolation by ANM was found to be sample independent. Both MCF-7 breast cancer cell CCM and human plasma EV isolation showed consistent results (FIG. 6C). EV loss may be correlated with the pressure as a result pore geometry in addition to fusion, which can increase pressure and high pressure by itself can induce fusion and lysing.

### Example 5

High-Yield Exosome Isolation with Tangential-Flow ANM Nano-Filtration Device

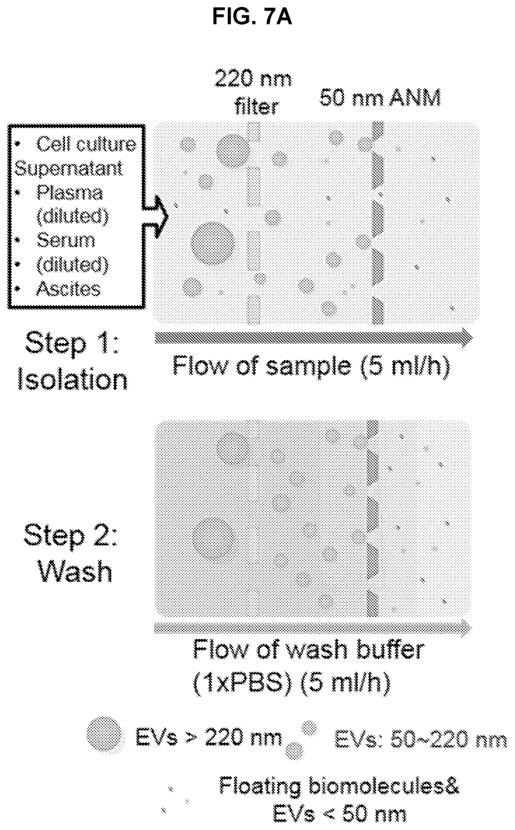

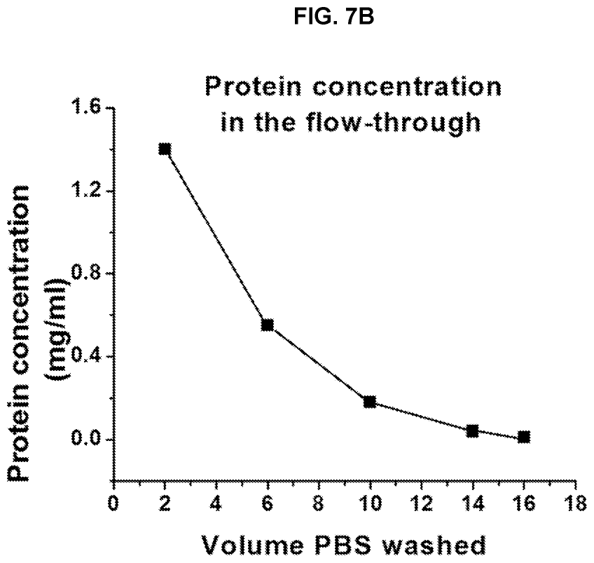

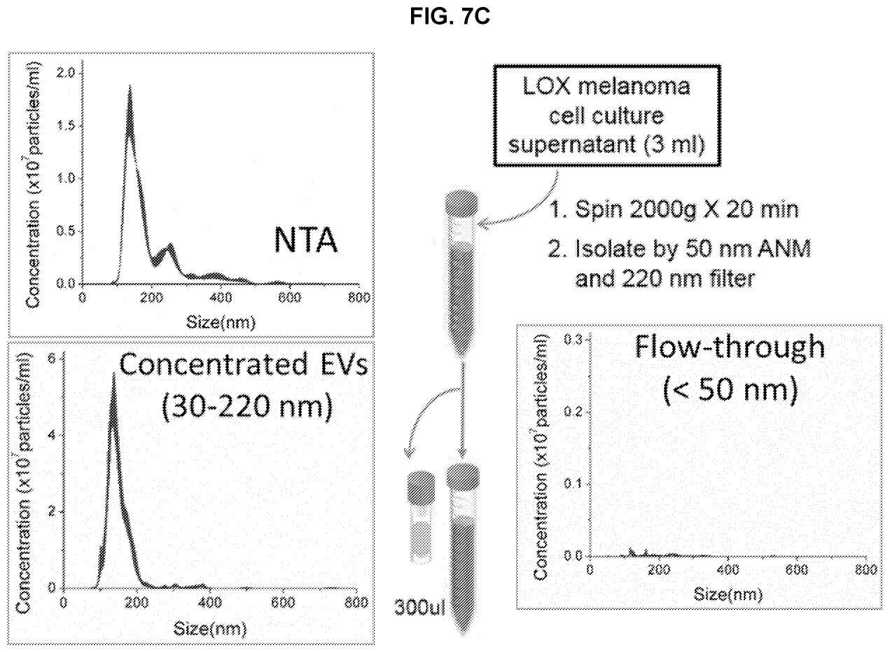

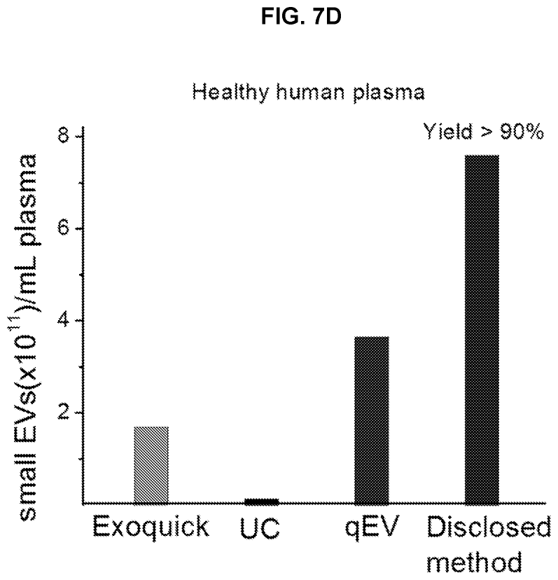

The tangential-flow ANM nano-filtration device was used to isolate exosomes from heterogeneous samples and characterize both the yield and purity of isolated exosomes. To isolate exosomes, the sample (cell culture supernatant or diluted plasma) was passed at a flow rate of 5 mL/h sequentially through a 200 nm ANM filtration cassette to filter out large EVs and then through a 50 nm ANM filtration cassette to isolate and concentrate the exosomes (FIG. 7A). A tangential-flow of buffer solution was introduced to prevent filter-cake formation on the ANM surface and clogging at the nanopore tip (FIG. 7A). This AMN filtration technology allowed for complete exosome and protein separation due to the presence of the 50 nm asymmetric nanopore filter and the additional buffer washing step for the trapped exosomes between the two membranes. FIG. 7B shows the protein concentration in the flow-through as a function of the washing buffer pumped through the device, following an exponential decay. Analysis of the isolated fractions by NTA showed successful separation of a mixture of particles (before isolation) into concentrated 30-220 nm EVs (after isolation; FIG. 7C). The exosome yield using the ANM was ˜10-20-fold higher than a size-exclusion chromatography method (qEV) and precipitation techniques (Exoquick-TC) and 10-fold higher than cylindrical nanopore membranes (FIG. 7E). A CD63-enriched 30-200 nm small EV subpopulation (herein referred to as the small EV or sEV fraction) was isolated. This fraction is devoid of protein markers of large EVs, LDL and exomeres, as well as classical exosomes. However, ApoA1 was detected in this sample, which indicated that HDL particles (slightly smaller than 30 nm) were still captured by ANM along with the sEVs. The purity will be further improved by eliminating HDLs in the isolated exosome population by using a baffled tangential-flow design as described above. The baffles create vortices in the tangential flow to prevent filter cake formation, which causes HDL entrapment. Two-stage ANM separation will also be used. Also, there was no observable sEVs in the flow-through which confirmed the efficiency of sEV capture using the AMN technology. The AMN-isolated sEV fraction will be characterized across various tumor cell lines and sera.

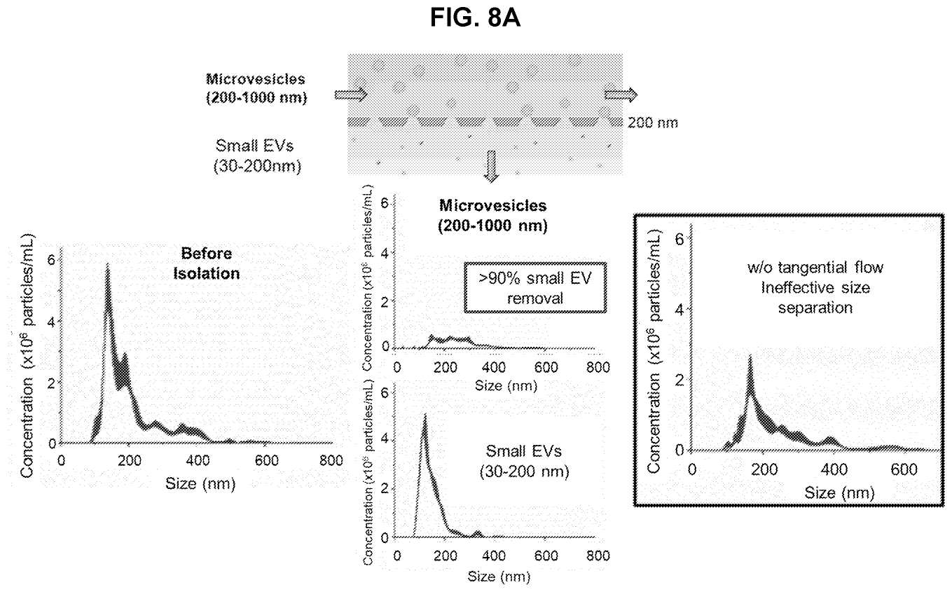

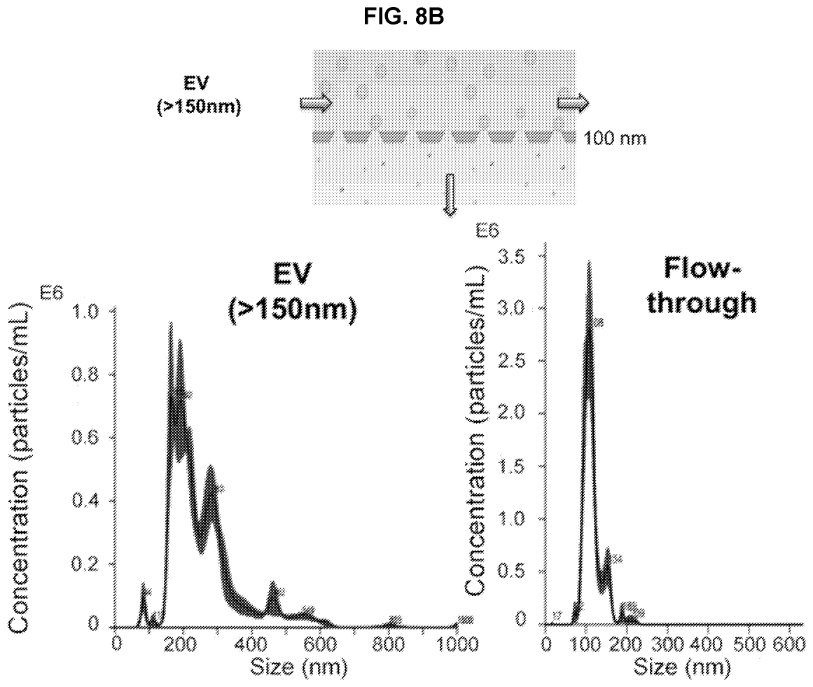

To fractionate the EVs into large EVs and sEVs, the sample was first passed through a 200 nm ANM filtration cassette to separate larger EVs, then through a 30 nm ANM filtration cassette to isolate the sEVs. Unlike the previous sEV isolation experiment, the EVs in the retentate from the 200 nm ANM filtration step was also collected and corresponded to the large EV fraction. The NTA profiles of isolated fractions indicated successful fractionation of heterogeneous EVs into large EVs and sEVs after isolation (FIG. 8A). In the large EVs fraction, 90% sEVs had been removed. The purity will be further improved by optimizing the tangential flow rate and washing buffer to allow for better size separation. The use of a 100 nm ANM instead of a 200 nm ANM may also allow for separation of EVs larger than 150 nm (FIG. 8B).

### Example 6

### Development of an ANM-Based Immunocapture Device

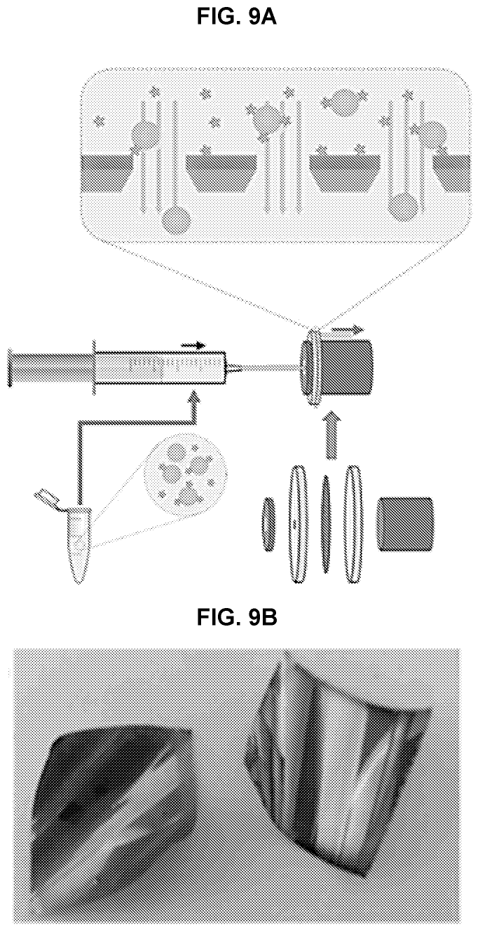

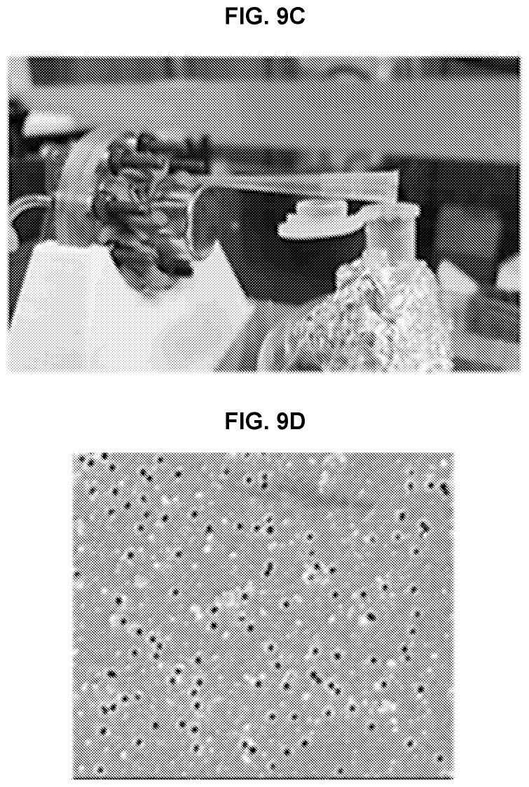

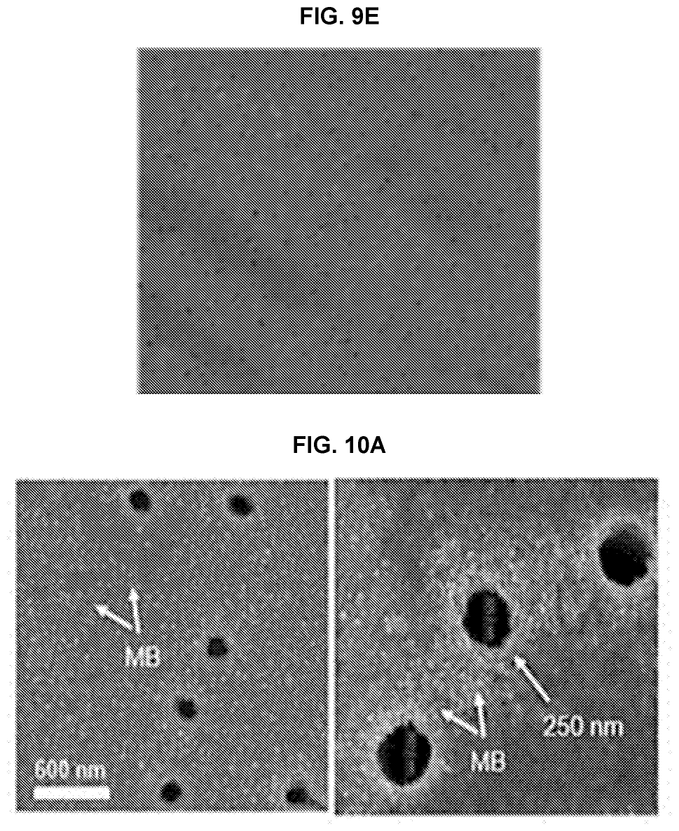

Immunocapture uses antibodies that target different surface proteins. Traditional immunocapture methods use magnetic beads at the micron size and bulk magnets to precipitate beads. The low diffusivity of these large beads leads to prolonged incubation that can take more than 24 hours. Magnetic nanoscale-sized beads on the other hand, can be captured in less than 1 hour. However, they are difficult to capture because of their paramagnetic nature. To address this, a protocol to coat the ANM with a layer of nickel-iron alloy was developed to make a Magnetic Nanopore Membrane (MNM) for fast immunocapture of specific carriers as shown in FIGS. 9A-FIG. 9E. The fabricated membranes are stable and can be easily integrated into the microfluidic platforms for rapid isolation of EVs, LLPs, and RNPs.

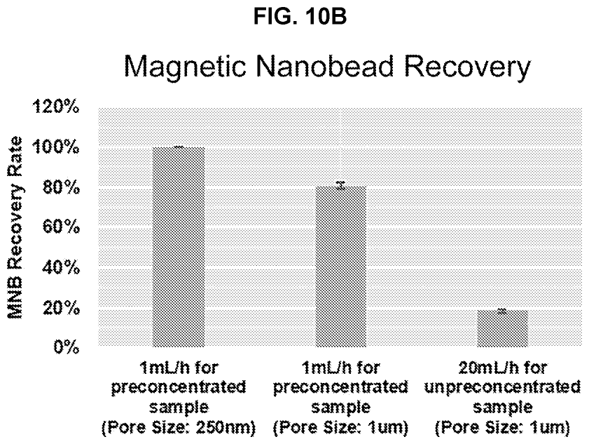

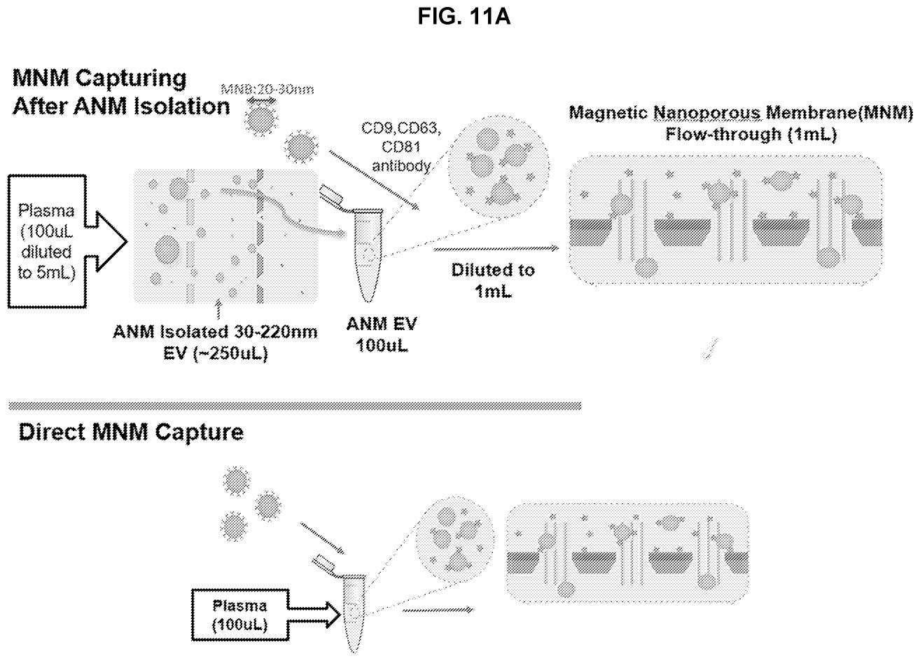

The recovery of Magnetic NanoBeads (MNB) with MNM was tested with 30 nm MNBs. FIG. 10A shows SEM images of MNBs captured near the pore entrances. A large portion of particles were captured at the edge of the pores, because of the high magnetic field gradient at the corners. NTA results suggest a more than 95% recovery rate by a membrane with a 250 nm pore size at a throughput of 1 mL/h (FIG. 10B). The yield decreased with increasing pore size and flow. When both large pores and a high flow rate were used, less than 20% of nanobeads were collected with the membrane. The need for small pores in the MNM means that the larger nanocarriers need to be removed prior to MNB capture or they will clog the MNM. This study hence highlights the benefits of a multi-stage design where an upstream ANM module is integrated with a downstream MNB and MNM immunocapture module.

### Example 7

Magnetic NanoBead/Nanopore Membrane (MNB/MNM) EV Fractionation from Plasma

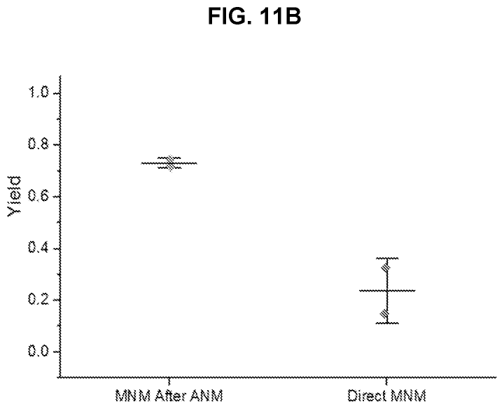

The yield of EV subgroup isolation with an MNM was tested with healthy human plasma. Both direct immunocapture of the plasma sample with no purification and immunocapture of EVs isolated and purified by ANM were examined (FIG. 11A-FIG. 11B). Briefly, 20 μL of nanomagnetic beads with a panel of exosome tetraspanin antibodies (CD9, CD63, CD81) were mixed with both 1 mL samples. After 30 minutes of incubation at room temperature, the mixture was passed through the MNM for 1 hour. NTA results showed that greater than 70% of the total EVs were captured with the pre-purified sample (FIG. 11B). However, only a 20% recovery rate was achieved in the direct MNM immunocapture. This may be due to interference of the immunocapture by other proteins in the original sample. Currently, it takes 90 minutes for the entire immunocapture process. To further shorten the fractionation process, a new microfluidic device integrated with both ANM and MNM sequentially in a continuous flow design will be developed. The raw sample will be first filtered by an upstream ANM module and mixed with an MNB stream. Mixing channels for agitation will be used to further accelerate the incubation process. The fully incubated mixture will pass through the capturing MNM module downstream. This immunocapture technology will also be developed for LLPs and RNPs and the modules can then be downstream stages.

What is claimed is:

1. A system for isolating exosomes comprising:
a first chamber;
a second chamber;
a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter;
a sample comprising exosomes positioned within the first chamber; and
a device for inducing fluid flow through the membrane from the first chamber to the second chamber by pressure driven flow, electroosmotic flow, centrifugal force, or a combination thereof.

2. The system of claim 1, wherein the first chamber comprises a wall opposite of the first membrane that comprises one or more baffles.

3. A system for isolating exosomes comprising:
a first chamber;
a second chamber;
a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter;
wherein the first chamber comprises a wall opposite of the first membrane that comprises one or more baffles;
a sample comprising exosomes positioned within the first chamber; and
a device for inducing fluid flow through the membrane from the first chamber to the second chamber by pressure driven flow, electroosmotic flow, centrifugal force, or a combination thereof.

4. The system of any one of claims 1-3, wherein the first membrane surface is coated with a magnetic alloy.

5. The system of any one of claims 1-3, wherein the first diameter is between about 10 nm and about 200 nm.

6. The system of any one of claims 1-5, wherein the second diameter is less than about 2 μm.

7. The system of any one of claims 1-6, wherein the membrane is formed from one or more materials comprising one or more of a polyethylene terephthalate (PET), a polycarbonate (PC), a polypropylene (PP), a polyimides (PI), or a polyethersulphone (PES).

8. The system of any one of claims 1-7, further comprising a third chamber and a filter positioned between the third chamber and the first chamber, the filter comprising a first filter surface facing and at least partially defining the third chamber, a second filter surface facing and at least partially defining the first chamber and a plurality of filter pores extending between the first and second filter surfaces.

9. The system of claim 8, wherein each filter pore has a diameter of 200 nm to 5 microns.

10. The system of either claim 8 or claim 9, wherein the filter is formed from one or more materials comprising a polyethylene terephthalate (PET), a polycarbonate (PC), a polypropylene (PP), a polyimides (PI), and a polyethersulphone (PES).

11. The system of any one of claims 1-3 or claims 5-10, further comprising a fourth chamber and a second membrane positioned between the fourth chamber and the second chamber, comprising a first membrane surface facing and at least partially defining the second chamber and a second membrane surface facing and at least partially defining the fourth chamber;
wherein the second membrane is the membrane of claim 21; and,
wherein the first membrane surface is coated with a magnetic alloy.

12. The system of any one of claims 1-11, wherein the device for inducing fluid flow generates a flow rate of between about 0.01 mL/hour to about 1000 mL/hour.

13. The system of any one of claim 1-12, wherein the device for inducing fluid flow generates a pressure less than about 1 atm.

14. The system of any one of claims 1-13, wherein the device for inducing fluid flow comprises a syringe pump, an electroosmotic pump, a micropump, a centrifuge, or a combination thereof.

15. The system of any one of claims 1-14, wherein the sample is applied perpendicularly or tangentially to the membrane or the filter.

16. The system of any one of claims 4-15, wherein the magnetic alloy is nickel-iron, samarium-cobalt, aluminum-nickel-cobalt, nickel-iron-chromium, iron-chromium-cobalt, or neodymium-iron-boron.

17. The system of any one of claims 4-16, wherein the exosomes are bound to a probe that is coupled to a magnetic bead.

18. The system of claim 17, wherein the probe is an antibody.

19. A method for isolating exosomes comprising:
providing the system of any of claims 1-18, and
inducing fluid flow through the membrane from the first chamber to the second chamber, whereupon the exosomes are isolated in the second chamber.

20. An exosome isolated using the method of claim 19.

21. A method for isolating exosomes comprising:
providing a system comprising:
a first chamber;
a second chamber;
a third chamber;
a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter;
a filter positioned between the third chamber and the first chamber, the filter comprising a first filter surface facing and at least partially defining the third chamber, a second filter surface facing and at least partially defining the first chamber and a plurality of filter pores extending between the first and second filter surfaces; and
a device for inducing fluid flow through the filter from the third chamber to the first chamber and through the membrane from the first chamber to the second chamber by pressure driven flow, electroosmotic flow, centrifugal force, or a combination thereof;
introducing a sample comprising exosomes into the third chamber;
inducing fluid flow through the filter and the membrane from the third chamber to the first chamber and from the first chamber to the second chamber, whereupon the exosomes pass through the filter and are isolated in the second chamber.

22. The system of claim 21, further comprising a fourth chamber and a second membrane positioned between the fourth chamber and the second chamber, comprising a first membrane surface facing and at least partially defining the second chamber and a second membrane surface facing and at least partially defining the fourth chamber;
wherein the second membrane is the membrane of claim 21; and,
wherein the first membrane surface is coated with a magnetic alloy.

23. The system of claim 21, wherein the first membrane surface is coated with a magnetic alloy.

24. The method of any one of claims 21-23, wherein the sample comprising exosomes comprises one or more of cell culture supernatants, a sample obtained from an animal subject, or an apoplastic fluid from a plant.

25. The method of any one of claims 21-24, wherein the sample obtained from an animal subject comprises one or more of blood, plasma, tear, serum, urine, sputum, pleural effusion, or ascites.

26. The method of any one of claims 21-25, wherein the first diameter is between about 10 nm to about 200 nm.

27. The method of any one of claims 21-26, wherein the second diameter is less than about 2 μm.

28. The method of any one of claims 21-27, wherein the membrane is formed from one or more materials comprising a polyethylene terephthalate (PET), a polycarbonate (PC), a polypropylene (PP), a polyimides (PI), or a polyethersulphone (PES).

29. The method of any one of claims 21-28, wherein each filter pore has a diameter of 200 nm to 5 microns.

30. The method of any one of claims 21-29, wherein the filter is formed from one or more materials comprising a polyethylene terephthalate (PET), a polycarbonate (PC), a polypropylene (PP), a polyimides (PI), or a polyethersulphone (PES).

31. The method of any one of claims 21-30, wherein the first chamber comprises a wall opposite of the first membrane that comprises one or more baffles.

32. The method of any one of claims 21-31, wherein the device for flowing the sample generates a flow rate of between about 0.01 mL/hour to about 1000 mL/hour.

33. The method of any one of claims 21-32, wherein the device for inducing fluid flow generates a pressure less than about 1 atm.

34. The method of any one of claims 21-33, wherein the device for inducing fluid flow comprises a syringe pump, an electroosmotic pump, a micropump, a centrifuge, or a combination thereof.

35. The method of any one of claims 21-34, wherein the sample is applied perpendicularly or tangentially to the filter.

36. The system of any one of claims 22-35, wherein the magnetic alloy is nickel-iron, samarium-cobalt, aluminum-nickel-cobalt, nickel-iron-chromium, iron-chromium-cobalt, or neodymium-iron-boron.

37. The system of any one of claims 22-36, wherein the exosomes are bound to a probe that is coupled to a magnetic bead.

38. The system of claim 37, wherein the probe is an antibody.

39. An exosome isolated using the method of any one of claims 21-38.
